(19) United States
(12) Patent Application Publication
(10) Pub. No.: US 2026/0200916 A1
(43) Pub. Date: Jul. 16, 2026

(54) 6,6 BICYCLIC HETEROCYLES USEFUL AS INHIBITORS OF NOD-LIKE RECEPTOR PROTEIN 3

(71) Applicant: Merck Sharp & Dohme LLC, Rahway, NJ (US)

(72) Inventors: Zachary G. BRILL, San Francisco, CA (US); Chen CHENG, Sunnyvale, CA (US); Donna A.A.W. HAYES, San Francisco, CA (US); Rohan Rajiv MERCHANT, San Francisco, CA (US); Essam METWALLY, Emerald Hills, CA (US); Anilkumar G NAIR, Morganville, NJ (US); Maoqun TIAN, Foster City, CA (US)

(73) Assignee: Merck Sharp & Dohme LLC, Rahway, NJ (US)

(21) Appl. No.: 19/137,741

(22) PCT Filed: Dec. 14, 2023
(86) PCT No.: PCT/US2023/083945

§ 371 Date: Jun. 11, 2025

Related U.S. Application Data
(60) Provisional application No. 63/433,533, filed on Dec. 19, 2022.

(51) Int. Cl. C07D 471/04; A61K 31/5025

(52) U.S. Cl. CPC C07D 471/04; A61K 31/5025

(57) ABSTRACT

Novel compounds of the structural Formula (I), and the pharmaceutically acceptable salts thereof are inhibitors of NLRP3 and may be useful in the treatment, prevention, management, amelioration, control and suppression of diseases mediated by NLPR3. The compounds of structural formula I may be useful in the treatment, prevention or management of diseases, disorders and conditions mediated by NLRP3 such as, but not limited to, gout, pseudogout, CAPS, NASH fibrosis, heart failure, idiophathic pericarditis, atopic dermatitis, inflammatory bowel disease, Alzheimer's Disease. Parkinson's Disease and traumatic brain injury.

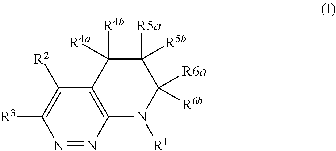

### CROSS REFERENCE TO RELATED APPLICATION

This application is an International Patent Application which claims priority from and the benefit of U.S. Provisional Application No. 63/433,533, filed Dec. 19, 2022; which is incorporated by reference in its entirety herein.

### BACKGROUND

Inflammasomes function as central signalling hubs of the innate immune system. They are multi-protein complexes assembled after activation of intracellular pattern recognition receptors (PRRs) by a variety of pathogen-associated molecular patterns (PAMPs) or danger-associated molecular patterns (DAMPs). It has been shown that inflammasomes can be formed by nucleotide-binding oligomerization domain (NOD)-like receptors (NLRs) and Pyrin and HIN200-domain-containing proteins (Van Opdenbosch N and Lamkanfi M. Immunity. 2019 Jun. 18; 50(6):1352-1364). Inflammasome activation triggers a cascade of events that releases pro-inflammatory cytokines, and promotes an inflammatory form of cell death called pyroptosis induced by the activation of Gasdermin. Pyroptosis is a unique form of inflammatory cell death that leads to the release of not only cytokines but also other intracellular components that promote a broader immune response both of the innate and acquired immune system. Thus, inflammasome activation is a major regulator of the inflammatory cascade.

The (NOD)-like receptor protein 3 (NLRP3) inflammasome is the most well-studied of all the inflammasomes. NLRP3 can be activated by numerous stimuli including environmental crystals, pollutants, host-derived DAMPs and protein aggregates (Tartey S and Kanneganti T D. Immunology, 2019 April; 156(4):329-338). Danger-associated molecular patterns that engage NLRP3 include uric acid and cholesterol crystals that cause gout and atherosclerosis, amyloid-P fibrils that are neurotoxic in Alzheimer's disease, and asbestos particles that cause mesothelioma (Kelley et al., Int J Mol Sci, 2019 Jul. 6; 20(13)). Additionally, NLRP3 is activated by infectious agents, such as Vibrio cholerae, fungal pathogens, such as Aspergillus Jumigatus and Candida albicans, adenoviruses, influenza A virus and SARS-CoV-2 (Tartey and Kanneganti, 2019 (see above); Fung et al. Emerg Microbes Infect, 2020 Mar. 14; 9(1):558-570).

The NLRP3 activation mechanism in humans remains unclear. It has been suggested that the NLRP3 inflammasome requires regulation at both the transcriptional and the post-transcriptional level (Yang Yet al., Cell Death Dis. 2019 Feb. 12; 10(2): 128). The NOD-like receptor protein 3 (NLRP3) is a protein-coding gene that encodes a protein consisting of a N-terminal pyrin domain, a nucleotide-binding site domain (NBD), and a leucine-rich repeat (LRR) motif on the C-terminal (Inoue et al., Immunology, 2013, 139, 11-18; Sharif et al., Nature, 2019 June; 570(7761):338-343).

In response to sterile inflammatory danger signals PAMPs or DAMPs, NLRP3 interacts with the adaptor protein, apoptosis-associated speck-like protein containing a caspase recruitment domain (ASC), and with the protease caspase-1 to form the NLRP3 inflammasome. Upon activation, procaspase-1 undergoes autoproteolysis and cleaves gasdermin D (Gsdmd) to produce the N-terminal Gsdmd molecule that leads to pore-formation in the plasma membrane and results in a lytic form of cell death called pyroptosis. Alternatively, caspase-1 cleaves the pro-inflammatory cytokines pro-IL-Iβ and pro-IL-18 to allow release of its biological active form (Kelley et al., 2019—see above). The NLRP3 inflammasome activation results in the release of the inflammatory cytokines IL-1β (interleukin-1β) and IL-18 (interleukin-18), which when dysregulated can lead to a number of diseases.

Dysregulation of the NLRP3 inflammasome or its downstream mediators are associated with numerous immune diseases, inflammatory diseases, auto-immune diseases and auto-inflammatory diseases. Activation of the NLRP3 inflammasome has been linked to the following diseases and disorders: Cryopyrin-associated Periodic Syndromes; sickle cell disease; systemic lupus erythematosus: allodynia; graft versus host disease; hepatic disorders including non-alcoholic steatohepatitis (NASH), chronic liver disease, viral hepatitis, alcoholic steatohepatitis, and alcoholic liver disease; inflammatory bowel diseases including Crohn's disease and ulcerative colitis; inflammatory joint disorders including gout, pseudogout, arthropathy, osteoarthritis, rheumatoid arthritis; additional rheumatic diseases including dermatomyositis, Still's disease, and juvenile idiopathic arthritis, kidney related diseases including hyperoxaluria, lupus nephritis, hypertensive nephropathy, hemodialysis related inflammation, diabetic nephropathy, and diabetic kidney disease and other inflammatory diseases (Miyamae T. Paediatr Drugs, 2012 Apr. 1, 14(2): 109-17: Szabo G and Petrasek J. Nat Rev Gastroenterol Hepatol, 2015 July; 12(7):387-400; Zhen Y and Zhang H. Front Immunol, 2019 Feb. 28:10:276; Vande Walle Let al., Nature, 2014 Aug. 7; 512(7512):69-73; Knauf et al., Kidney Int, 2013 November; 84(5):895-901; Krishnan et al., Br J Pharmacol, 2016 February; 1 73(4):752-65); Shahzad et al., Kidney Int, 2015 January; 87(1):74-84; Jankovic, et al. J Exp Med. 2013 Sep. 23; 210(10):1899-910.). The onset and progression of neuroinflammation-related disorders, such as brain infection, acute injury, multiple sclerosis, amyotrophic lateral sclerosis and additional neurodegenerative diseases such as Parkinsons and Alzheimer's disease have also been linked to NLRP3 inflammasome activation (Sarkar et al., NPJ Parkinsons Dis, 2017 Oct. 17:3:30).

Cardiovascular and metabolic disorders such as atherosclerosis, type I and type II diabetes and diabetes complications including nephropathy and retinopathy, peripheral artery disease, acute heart failure and hypertension have been associated to NLRP3 (Ridker et al., CANTOS Trial Group. N Engl J Med. 2017 Sep. 21; 377(12):1119-1131; and Toldo S and Abbate A Nat Rev Cardiol, 2018 April; 15(4):203-214). NLRP3 associated skin diseases include wound healing and scar formation; inflammatory skin diseases such as acne, atopic dermatitis, hidradenitis suppurativa and psoriasis (Kelly et al., Br J Dermatol, 2015 December; 1 73(6)). NLRP3 inflammasome activity has also been linked to respiratory conditions such as asthma, sarcoidosis, acute respiratory distress syndrome, Severe Acute Respiratory Syndrome (SARS) (Nieto-Torres et al., Virology. 2015 November: 485:330-9)); and ocular diseases including age-related macular degeneration (AMD) and diabetic retinopathy (Doyle et al., Nat Med, 2012 May; 18(5):791-8). Cancers linked to NLRP3 include myeloproliferative neoplasms, leukemias, myelodysplastic syndromes, myelofibrosis, lung cancer and colon cancer (Ridker et al., Lancet, 2017 Oct. 21; 390(10105): 1833-1842; Derangere et al., Cell Death Differ. 2014 December; 21(12): 1914-24; Basiorka et al., Lancet Haematol, 2018 September; 5(9): e393-e402, Zhang et al., Hum Immunol, 2018 January; 79(1):57-62).

Immune diseases and inflammatory disorders are typically difficult to diagnose or treat efficiently and effectively. Most treatments include treatment of the symptoms, slowing down disease progression, lifestyle changes and surgery.

There remains a need for inhibitors of NLRP3 to provide new treatments for diseases and disorders associated with NLRP3 inflammasome activation and dysregulation. The compounds of structural formula I are useful for the treatment and prevention of diseases, disorders and conditions mediated by formation and propogation of the NLRP3 inflammasome.

NLRP3 inhibitors are disclosed in the following publications: Nat. 2022, 1; Cell. 2021, 184, 1; J Mol. Biol. 2021, 433, 167308; J Med. Chem. 2021, 64, 101; Nat. Chem. Biol. 2019, 15, 556; Nat. 2019, 570, 338; Nat. Chem. Biol. 2019, 15, 560; PLOS Biol. 2019, 1; Nat. Med. 2015, 21, 248; Cell. 2014, 156, 1193; Nat. Immunol. 2014, 15, 738; PNAS. 2007, 104, 8041; Nat. 2006, 440, 9; Immunity. 2006, 24, 317. Several patent applications describe NLRP3 inhibitors, including WO 2021/239885, WO 2021/209552, WO 2021/209539, WO 2021/193897, WO 2020/018975, WO 2020/037116, WO 2020/021447, WO 2020/010143, WO 2019/079119. WO 2019/0166621. WO 2019/121691. WO 2019/034696. WO 2019/034697, WO 2019/034693, WO 2019/034692, WO 2019/034690, WO 2019/034688, WO 2019/034686, WO 2019/008025. WO 2019/008029, WO 2019/023145, WO 2019/023147, WO 2019/025467, WO 2018/167468, WO 2018/015445, WO 2017/184746, WO 2017/184735, WO 2017/184623, WO 2017/184604. WO 2017/184624, WO 2017/140778, WO 2016/131098, U.S. Pat. No. 11,319,319, and US 2020/0361898.

### SUMMARY

The present disclosure relates to novel compounds of structural formula I:

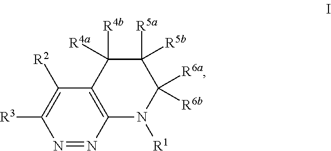

and pharmaceutically acceptable salts thereof.

The compounds of structural formula I, and embodiments thereof, are inhibitors ofNOD-like receptor protein 3 (NLRP3) and may be useful in the treatment and prevention of diseases, disorders and conditions mediated by NLRP3 such as, but not limited to, gout, pseudogout (chondrocalcinosis), cryopyrin-associated periodic syndromes (CAPS), NASH, fibrosis, heart failure, idiophathic pericarditis, atopic dermatitis, inflammatory bowel disease, Alzheimer's Disease, Parkinson's Disease and traumatic brain injury.

The present disclosure also relates to pharmaceutical compositions comprising the compounds of structural formula I and a pharmaceutically acceptable carrier.

Also disclosed are methods for the treatment, management, prevention, alleviation, amelioration, suppression or control of disorders, diseases, and conditions that may be responsive to inhibition of the NLRP3 receptor in a subject in need thereof by administering the compounds and pharmaceutical compositions of the present disclosure.

The present disclosure also relates to the use of compounds of structural formula I for manufacture of a medicament useful in treating diseases, disorders and conditions that may be responsive to the inhibition of the NLRP3 receptor.

The present disclosure is also concerned with treatment or prevention of these diseases, disorders and conditions by administering the compounds of structural formula I in combination with a therapeutically effective amount of another agent that may be useful to treat the disease, disorder and condition. The disclosure is further concerned with processes for preparing the compounds of structural formula I.

### DETAILED DESCRIPTION

The present disclosure is concerned with novel compounds of structural Formula I:

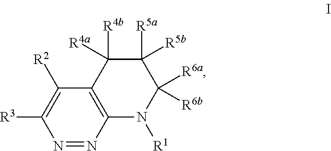

or a pharmaceutically acceptable salt thereof, wherein
R¹ is selected from the group:
1) —C₃₋₁₂cycloalkyl,
2) —C₃₋₁₂cycloalkenyl,
3) —C₂₋₁₁cycloheteroalkyl,
4) —C₂₋₁₁cycloheteroalkenyl,
5) aryl,
6) heteroaryl,
7) —C₁₋₆alkyl.
8) —C₁₋₆alkyl-OH,
9) —C₁₋₆alkyl-C₃₋₁₂cycloalkyl.
10) —C₁₋₆alkyl-C₂₋₁₁cycloheteroalkyl,
11) —C₁₋₆alkyl-C₂₋₁₁cycloheteroalkenyl,
12) —C₁₋₆alkyl-aryl, and
13) —C₁₋₆alkyl-heteroaryl,
wherein each alkyl, cycloalkyl, cycloalkenyl, cycloheteroalkyl, cycloheteroalkenyl, aryl and heteroaryl is unsubstituted or substituted with one to six substituents selected from R^{a};
R² is selected from the group:
1) hydrogen,
2) CN,
3) —CF₃,
4) —CHF₂,
5) —C₁₋₆alkyl, and
6) halogen,
wherein alkyl is unsubstituted or substituted with one to five substituents selected from R^{b};
R³ is selected from the group:
1) aryl, and
2) heteroaryl,
wherein each aryl and heteroaryl is unsubstituted or substituted with one to five substituents selected from R^{c};
R^{4a} is selected from the group:
1) hydrogen,
2) CN,
3) —C₁₋₆alkyl,
4) —O—C₁₋₆alkyl,
5) —N(R^{k})₂, and
6) halogen,
wherein each alkyl is unsubstituted or substituted with one to five substituents selected from R^{d};
R^{4b} is selected from the group:
1) hydrogen,
2) CN,
3) —C₁₋₆alkyl,
4) —O—C₁₋₆alkyl,
5) —N(R^{k})₂, and
6) halogen,
wherein each alkyl is unsubstituted or substituted with one to five substituents selected from R^{d};
R^{5a} is selected from the group:
1) hydrogen,
2) CN,
3) —C₁₋₆alkyl,
4) —O—C₁₋₆alkyl,
5) —N(R^{k})₂,
6) halogen,
7) —C₃₋₆cycloalkyl, and
8) —C₂₋₆cycloheteroalkyl,
wherein each alkyl, cycloalkyl and cycloheteroalkyl is unsubstituted or substituted with one to five substituents selected from R^{e};
R^{5b} is selected from the group:
1) hydrogen,
2) CN,
3) —C₁₋₆alkyl,
4) —O—C₁₋₆alkyl,
5) —N(R^{k})₂,
6) halogen,
7) —C₃₋₆cycloalkyl, and
8) —C₂₋₆cycloheteroalkyl,
wherein each alkyl, cycloalkyl and cycloheteroalkyl is unsubstituted or substituted with one to five substituents selected from R^{e}:
R^{6a} is selected from the group:
1) hydrogen,
2) CN,
3) —C₁₋₆alkyl, and
4) halogen,
wherein each alkyl is unsubstituted or substituted with one to five substituents selected from R^{e};
R^{6b} is selected from the group:
1) hydrogen,
2) CN,
3) —C₁₋₆alkyl, and
4) halogen,
wherein each alkyl is unsubstituted or substituted with one to five substituents selected from R^{e};
each R^{a} is independently selected from the group:
1) CN,
2) oxo,
3) —OH,
4) halogen,
5) —C₁₋₆alkyl,
6) —C₁₋₆alkyl-OH,
7) —O—C₁₋₆alkyl,
8) —C₃₋₆cycloalkyl,
9) —C₂₋₆cycloheteroalkyl,
10) aryl,
11) heteroaryl,
12) —C(O)C₁₋₆alkyl,
13) —C(O)C₃₋₆cycloalkyl,
14) —C₁₋₆alkyl-aryl,
15) —C₁₋₆alkyl-heteroaryl,
16) —C₁₋₆alkyl-C₃₋₆cycloalkyl,
17) —C₁₋₆alkyl-C₂₋₆cycloheteroalkyl,
18) —(CH₂)ₚ—O—C₁₋₆alkyl,
19) —(CH₂)ₚ—O—C₃₋₆cycloalkyl,
20) —(CH₂)ₚ—O—C₂₋₆cycloheteroalkyl.
21) —(CH₂)ₚ—O-aryl,
22) —(CH₂)ₚ—O-heteroaryl.
23) —(CH₂)ₚ—S(O)ᵣR^{f}, and
24) —N(R^{g})₂,
wherein each alkyl, cycloalkyl, cycloheteroalkyl, aryl and heteroaryl is unsubstituted or substituted with one to six substituents selected from halogen, CF₃, OH, C₁₋₆alkyl, and —OC₁₋₆-alkyl;
each R^{b} is independently selected from the group:
1) CF₃,
2) halogen,
3) —C₁₋₆alkyl, and
4) —C₃₋₆cycloalkyl;
each R^{c} is independently selected from the group:
1) CN,
2) CF₃,
3) —OH,
4) oxo,
5) halogen,
6) —C₁₋₆alkyl,
7) —O—C₁₋₆alkyl,
8) —C₃₋₆cycloalkyl,
9) —C₂₋₆cycloheteroalkyl,
10) aryl,
11) heteroaryl,
12) —C₁₋₆alkyl-aryl,
13) —C₁₋₆alkyl-heteroaryl.
14) —C₁₋₆alkyl-C₃₋₆cycloalkyl,
15) —C₁₋₆alkyl-C₂₋₆cycloheteroalkyl,
16) —(CH₂)_{q}—O—C₁₋₆alkyl,
17) —(CH₂)_{q}—O—C₃₋₆cycloalkyl,
18) —(CH₂)_{q}—O—C₂₋₆cycloheteroalkyl,
19) —(CH₂)_{q}—O-aryl.
20) (CH₂)_{q}—O-heteroaryl,
21) —OC₁₋₆alkyl-C₃₋₆cycloalkyl,
22) —OC₁₋₆alkyl-C₂₋₆cycloheteroalkyl,
23) —OC₁₋₆alkyl-aryl,
24) —OC₁₋₆alkyl-heteroaryl,
25) —(CH₂)_{q}-S(O)ᵣR^{h}.
26) —N(Rⁱ)₂,
27) —C(O)R^{j}, and
28) —C(O)NRⁱ,
wherein each alkyl, cycloalkyl, cycloheteroalkyl, aryl, and heteroaryl is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl;
each R^{d} is independently selected from the group:
1) hydrogen,
2) OH
3) halogen, and
4) —C₁₋₆alkyl;
each R^{e} is independently selected from the group:
1) hydrogen,
2) OH,
3) halogen, and
4) —C₁₋₆alkyl:
each R^{f} is independently selected from the group:
1) hydrogen,
2) —C₁₋₆alkyl,
3) —C₃₋₆cycloalkyl, and
4) —C₂₋₆cycloheteroalkyl;
each R^{g} is independently selected from the group:
1) hydrogen,
2) —C₁₋₆alkyl.
3) —C₃₋₆cycloalkyl,
4) —C₂₋₆cycloheteroalkyl.
5) aryl,
6) heteroaryl,
7) —C(O)C₁₋₆alkyl, and
8) —S(O)ᵣR^{f},
wherein alkyl, cycloalkyl, cycloheteroalkyl, aryl and heteroaryl is unsubstituted or substituted with one to three substituents selected from: CF₃, halogen, OH and —OC₁₋₆alkyl;
each R^{h} is independently selected from the group:
1) hydrogen,
2) —C₁₋₆alkyl,
3) —C₃₋₆cycloalkyl, and
4) —C₂₋₆cycloheteroalkyl;
each Rⁱ is independently selected from the group:
1) hydrogen,
2) —C₁₋₆alkyl,
3) —C₃₋₆cycloalkyl, and
4) —C₂₋₆cycloheteroalkyl;
each R^{j} is independently selected from the group:
1) OH,
2) —C₁₋₆alkyl.
3) —C₃₋₆cycloalkyl, and
4) —C₂₋₆cycloheteroalkyl.
wherein alkyl, cycloalkyl and cycloheteroalkyl is unsubstituted or substituted with one to three substituents selected from: CF₃, halogen, OH and —OC₁₋₆alkyl:
each R^{k} is independently selected from the group:
1) hydrogen, and
2) —C₁₋₆alkyl;
p is 0, 1, 2, 3, 4, 5 or 6:
q is 0, 1, 2, 3, 4, 5 or 6; and
r is 1 or 2.

The disclosure has numerous embodiments, which are summarized below. The disclosure includes the compounds as shown, and also includes individual diastereoisomers, enantiomers, and epimers of the compounds, and mixtures of diastereoisomers and/or enantiomers thereof including racemic mixtures.

In one embodiment, R¹ is selected from the group: —C₃₋₁₂cycloalkyl, —C₃-12cycloalkenyl, —C₂₋₁₁cycloheteroalkyl, —C₂₋₁₁cycloheteroalkenyl, aryl, heteroaryl, —C₁₋₆alkyl, —C₁₋₆alkyl-OH, —C₁₋₆alkyl-C₃₋₁₂cycloalkyl, —C₁₋₆alkyl-C₂₋₁₁ cycloheteroalkyl, —C₁₋₆alkyl-C₂₋₁₁ cycloheteroalkenyl, —C₁₋₆alkyl-aryl, and —C₁₋₆alkyl-heteroaryl, wherein each alkyl, cycloalkyl, cycloalkenyl, cycloheteroalkyl, cycloheteroalkenyl, aryl and heteroaryl is unsubstituted or substituted with one to six substituents selected from R^{a}. In a class of this embodiment, Rⁱ is unsubstituted or substituted with one to four substituents selected from R^{a}. In another class of this embodiment, Rⁱ is unsubstituted or substituted with one to three substituents selected from R^{a}.

In another embodiment, R¹ is selected from the group: —C₃₋₁₂cycloalkyl, —C₂₋₁₁ cycloheteroalkyl, aryl, heteroaryl. —C₁₋₆alkyl, —C₁₋₆alkyl-OH, —C₁₋₆alkyl-C₃₋₁₂cycloalkyl, and —C₁₋₆alkyl-C₂₋₁₁cycloheteroalkyl, wherein each alkyl, cycloalkyl, cycloheteroalkyl, aryl and heteroaryl is unsubstituted or substituted with one to six substituents selected from R^{a}. In a class of this embodiment. Rⁱ is unsubstituted or substituted with one to four substituents selected from R^{a}. In another class of this embodiment, Rⁱ is unsubstituted or substituted with one to three substituents selected from R^{a}.

In another embodiment, R¹ is selected from the group: —C₃₋₁₂cycloalkyl, —C₂₋₁₁ cycloheteroalkyl, aryl, heteroaryl, —C₁₋₆alkyl-C₃₋₁₂cycloalkyl, and —C₁₋₆alkyl-C₂₋₁₁ cycloheteroalkyl, wherein each alkyl, cycloalkyl, cycloheteroalkyl, aryl and heteroaryl is unsubstituted or substituted with one to six substituents selected from R^{a}. In a class of this embodiment, R¹ is unsubstituted or substituted with one to four substituents selected from R^{a}. In another class of this embodiment, R¹ is unsubstituted or substituted with one to three substituents selected from R^{a}.

In another embodiment, R¹ is selected from the group: —C₃₋₁₂cycloalkyl, —C₂₋₁₁ cycloheteroalkyl, —C₁₋₆alkyl-C₃₋₁₂cycloalkyl, and —C₁₋₆alkyl-C₂₋₁₁cycloheteroalkyl, wherein each alkyl, cycloalkyl, and cycloheteroalkyl is unsubstituted or substituted with one to six substituents selected from R^{a}. In a class of this embodiment, R¹ is unsubstituted or substituted with one to four substituents selected from R^{a}. In another class of this embodiment, R¹ is unsubstituted or substituted with one to three substituents selected from R^{a}.

In another embodiment, R¹ is selected from the group: —C₃₋₁₂cycloalkyl, —C₂₋₁₁ cycloheteroalkyl, and —C₁₋₆alkyl-C₂₋₁₁ cycloheteroalkyl, wherein each alkyl, cycloalkyl, and cycloheteroalkyl is unsubstituted or substituted with one to six substituents selected from R^{a}. In a class of this embodiment, R¹ is unsubstituted or substituted with one to four substituents selected from R^{a}. In another class of this embodiment, R¹ is unsubstituted or substituted with one to three substituents selected from R^{a}. In another class of this embodiment, R¹ is selected from the group: cyclobutane, cyclohexane, bicyclo[3.1.1]heptane, piperidine, and —CH₂-pyrrolidine, wherein R¹ is unsubstituted or substituted with one to six substituents selected from R^{a}.

In another embodiment, R¹ is selected from the group: —C₃₋₁₂cycloalkyl, and —C₂₋₁₁ cycloheteroalkyl, wherein each cycloalkyl and cycloheteroalkyl is unsubstituted or substituted with one to six substituents selected from R^{a}. In a class of this embodiment, R¹ is unsubstituted or substituted with one to four substituents selected from R^{a}. In another class of this embodiment. R¹ is unsubstituted or substituted with one to three substituents selected from R^{a}. In another class of this embodiment, R¹ is selected from the group: cyclobutane, cyclohexane, bicyclo[3.1.1]heptane, and piperidine, wherein R¹ is unsubstituted or substituted with one to six substituents selected from R^{a}. In another class of this embodiment, R¹ is selected from the group: cyclobutane, and piperidine, wherein R¹ is unsubstituted or substituted with one to six substituents selected from R^{a}.

In another embodiment, R¹ is selected from the group: —C₃₋₁₂cycloalkyl, wherein cycloalkyl is unsubstituted or substituted with one to six substituents selected from R^{a}. In a class of this embodiment, R¹ is unsubstituted or substituted with one to four substituents selected from R^{a}. In another class of this embodiment. R¹ is unsubstituted or substituted with one to three substituents selected from R^{a}. In another class of this embodiment, R¹ is selected from the group: cyclobutane, cyclohexane, and bicyclo[3.1.1]heptane, wherein R¹ is unsubstituted or substituted with one to six substituents selected from R^{a}. In another class of this embodiment, R¹ is selected from the group: cyclohexane, and bicyclo[3.1.1]heptane, wherein R¹ is unsubstituted or substituted with one to six substituents selected from R^{a}. In another class of this embodiment, R¹ is cyclobutane, wherein R¹ is unsubstituted or substituted with one to six substituents selected from R^{a}.

In another embodiment, R¹ is —C₂₋₁₁cycloheteroalkyl, wherein cycloheteroalkyl is unsubstituted or substituted with one to six substituents selected from R^{a}. In a class of this embodiment, R¹ is unsubstituted or substituted with one to four substituents selected from R^{a}. In another class of this embodiment, R¹ is unsubstituted or substituted with one to three substituents selected from R^{a}. In another class of this embodiment, R¹ is piperidine, wherein R¹ is unsubstituted or substituted with one to six substituents selected from R^{a}.

In another embodiment, R² is selected from the group: hydrogen, CN, —CF₃, —CHF2, —C₁₋₆ alkyl, and halogen, wherein alkyl is unsubstituted or substituted with one to five substituents selected from R^{b}. In a class of this embodiment, alkyl is unsubstituted or substituted with one to four substituents selected from R^{b}. In another class of this embodiment, alkyl is unsubstituted or substituted with one to three substituents selected from R^{b}.

In another embodiment, R² is selected from the group: hydrogen, —CF₃. —CHF2, and —C₁₋₆ alkyl, wherein alkyl is unsubstituted or substituted with one to five substituents selected from R^{b}. In a class of this embodiment, alkyl is unsubstituted or substituted with one to four substituents selected from R^{b}. In another class of this embodiment, alkyl is unsubstituted or substituted with one to three substituents selected from R^{b}.

In another embodiment, R² is selected from the group: hydrogen, and —C₁₋₆alkyl, wherein alkyl is unsubstituted or substituted with one to five substituents selected from R^{b}. In a class of this embodiment, alkyl is unsubstituted or substituted with one to four substituents selected from R^{b}. In another class of this embodiment, alkyl is unsubstituted or substituted with one to three substituents selected from R^{b}.

In another class of this embodiment. R² is selected from the group: hydrogen, and —CH₃.

In another embodiment, R³ is selected from the group: aryl, and heteroaryl, wherein each aryl and heteroaryl is unsubstituted or substituted with one to five substituents selected from R^{c}. In a class of this embodiment, each aryl and heteroaryl is unsubstituted or substituted with one to four substituents selected from R^{c}. In another class of this embodiment, each aryl and heteroaryl is unsubstituted or substituted with one to three substituents selected from R^{c}.

In another embodiment, R³ is selected from the group: phenyl, benzofuran, and pyridine, wherein R³ is unsubstituted or substituted with one to five substituents selected from R^{c}. In a class of this embodiment, R³ is unsubstituted or substituted with one to four substituents selected from R^{c}. In another class of this embodiment. R³ is unsubstituted or substituted with one to three substituents selected from R^{c}.

In another embodiment, R³ is selected from the group: aryl, wherein aryl is unsubstituted or substituted with one to five substituents selected from R^{e}. In a class of this embodiment, R³ is unsubstituted or substituted with one to four substituents selected from R^{c}. In another class of this embodiment, R³ is unsubstituted or substituted with one to three substituents selected from R^{c}. In another class of this embodiment, R³ is phenyl, wherein phenyl is unsubstituted or substituted with one to five substituents selected from R^{c}. In a subclass of this class, R³ is unsubstituted or substituted with one to four substituents selected from R^{c}. In another subclass of this class, R³ is unsubstituted or substituted with one to three substituents selected from R^{e}.

In another embodiment, R³ is heteroaryl, wherein heteroaryl is unsubstituted or substituted with one to five substituents selected from R^{c}. In a class of this embodiment, R³ is unsubstituted or substituted with one to four substituents selected from R^{e}. In another class of this embodiment, R³ is unsubstituted or substituted with one to three substituents selected from R^{c}. In another class of this embodiment, R³ is selected from the group: benzofuran, and pyridine, wherein R³ is unsubstituted or substituted with one to five substituents selected from R^{c}.

In another embodiment, R^{4a} is selected from the group: hydrogen. CN, —C₁₋₆alkyl, —O—C₁₋₆alkyl, —N(R^{k})₂, and halogen, wherein each alkyl is unsubstituted or substituted with one to five substituents selected from R^{d}. In a class of this embodiment, alkyl is unsubstituted or substituted with one to four substituents selected from R^{d}. In another class of this embodiment, alkyl is unsubstituted or substituted with one to three substituents selected from R^{d}.

In another embodiment, R^{4a} is selected from the group: hydrogen, and —C₁₋₆alkyl, wherein each alkyl is unsubstituted or substituted with one to five substituents selected from R^{d}. In a class of this embodiment, alkyl is unsubstituted or substituted with one to four substituents selected from R^{d}. In another class of this embodiment, alkyl is unsubstituted or substituted with one to three substituents selected from R^{d}.

In another embodiment, R^{4a} is hydrogen.

In another embodiment, R^{4b} is selected from the group: hydrogen, CN, —C₁₋₆alkyl, —O—C₁₋₆alkyl, —N(R^{k})₂, and halogen, wherein each alkyl is unsubstituted or substituted with one to five substituents selected from R^{d}. In a class of this embodiment, alkyl is unsubstituted or substituted with one to four substituents selected from R^{d}. In another class of this embodiment, alkyl is unsubstituted or substituted with one to three substituents selected from R^{d}.

In another embodiment, R^{4b} is selected from the group: hydrogen, and —C₁₋₆alkyl, wherein each alkyl is unsubstituted or substituted with one to five substituents selected from R^{d}. In a class of this embodiment, alkyl is unsubstituted or substituted with one to four substituents selected from R^{d}. In another class of this embodiment, alkyl is unsubstituted or substituted with one to three substituents selected from R^{d}.

In another embodiment, R^{4b} is hydrogen.

In another embodiment, R^{5a} is selected from the group: hydrogen. CN, —C₁₋₆alkyl. —O—C₁₋₆alkyl, —N(R^{k})₂, halogen, —C₃₋₆cycloalkyl, and —C₂₋₆cycloheteroalkyl, wherein each alkyl, cycloalkyl, and cycloheteroalkyl is unsubstituted or substituted with one to five substituents selected from R^{e}. In a class of this embodiment. R^{5a} is unsubstituted or substituted with one to four substituents selected from R^{e}. In another class of this embodiment, R⁵, is unsubstituted or substituted with one to three substituents selected from R^{e}.

In another embodiment, R^{5a} is selected from the group: hydrogen. CN, —C₁₋₆alkyl, —O—C₁₋₆alkyl, —N(R^{k})₂, and halogen, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{5a} is selected from the group: hydrogen, —C₁₋₆alkyl, and —N(R^{k})₂, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{5a} is selected from the group: hydrogen, and —N(R^{k})₂.

In a class of this embodiment, R^{5a} is selected from the group: hydrogen and —NH2.

In another embodiment, R^{5b} is selected from the group: hydrogen, CN, —C₁₋₆alkyl, —O—C₁₋₆alkyl, —N(R^{k})₂, halogen, —C₃₋₆cycloalkyl, and —C₂₋₆cycloheteroalkyl, wherein each alkyl, cycloalkyl, and cycloheteroalkyl is unsubstituted or substituted with one to five substituents selected from R^{e}. In a class of this embodiment. R^{5b} is unsubstituted or substituted with one to four substituents selected from R^{e}. In another class of this embodiment. R⁵¹ is unsubstituted or substituted with one to three substituents selected from R^{e}.

In another embodiment, R^{5b} is selected from the group: hydrogen. CN, —C₁₋₆alkyl, —O—C₁₋₆alkyl, —N(R^{k})₂, and halogen, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{5b} is selected from the group: hydrogen, —C₁₋₆alkyl, and —N(R^{k})₂, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{5b} is selected from the group: hydrogen, and —N(R^{k})₂. In a class of this embodiment, R^{5b} is selected from the group: hydrogen, and —NH2.

In another embodiment, R^{6a} is selected from the group: hydrogen. CN, —C₁₋₆alkyl, and halogen, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{6a} is selected from the group: hydrogen, and —C₁₋₆alkyl, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{6a} is —C₁₋₆alkyl, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{6a} is hydrogen.

In another embodiment, R^{6b} is selected from the group: hydrogen, CN, —C₁₋₆alkyl, and halogen, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{6b} is selected from the group: hydrogen, and —C₁₋₆alkyl, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{6b} is —C₁₋₆alkyl, wherein each alkyl is unsubstituted or substituted with one to five substituents selected from R^{e}. In a class of this embodiment, alkyl is unsubstituted or substituted with one to four substituents selected from R^{e}. In another class of this embodiment, alkyl is unsubstituted or substituted with one to three substituents selected from R^{e}.

In another embodiment, R^{6b} is hydrogen.

In another embodiment, each R^{a} is independently selected from the group: CN, oxo, —OH, halogen, —C₁₋₆alkyl. —C₁₋₆alkyl-OH, —O—C₁₋₆alkyl. —C₃₋₆cycloalkyl. —C₂₋₆cycloheteroalkyl, aryl, heteroaryl, —C(O)C₁₋₆alkyl, —C(O)C₃₋₆cycloalkyl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —(CH₂)ₚ—O—C₁₋₆alkyl, —(CH₂)ₚ—O—C₃₋₆cycloalkyl, —(CH₂)ₚ—O—C₂₋₆cycloheteroalkyl, —(CH₂)ₚ—O-aryl, —(CH₂)ₚ—O-heteroaryl, —(CH₂)ₚ—S(O)ᵣR^{f}, and —N(R^{g})₂, wherein each alkyl, cycloalkyl, cycloheteroalkyl, aryl, and heteroaryl is unsubstituted or substituted with one to six substituents selected from halogen, CF₃, OH, C₁₋₆alkyl, and —OC₁₋₆alkyl. In a class of this embodiment, each R^{a} is independently selected from the group: CN, oxo, —OH, halogen. —C₁₋₆alkyl, —C₁₋₆alkyl-OH. —O—C₁₋₆alkyl, —C₃₋₆cycloalkyl, —C₂₋₆cycloheteroalkyl. —C(O)C₁₋₆alkyl. —C(O)C₃₋₆cycloalkyl, —C₁₋₆alkyl-C₃₋₆cycloalkyl, —C₁₋₆alkyl-C₂₋₆cycloheteroalkyl, —(CH₂)ₚ—O—C₁₋₆alkyl, (CH₂)ₚ—O—C₃₋₆cycloalkyl, —(CH₂)ₚ—O—C₂₋₆cycloheteroalkyl, —(CH₂)ₚ—S(O)ᵣR^{f}, and —N(R^{g})₂, wherein each alkyl, cycloalkyl, and cycloheteroalkyl is unsubstituted or substituted with one to six substituents selected from halogen, CF₃, OH. C₁₋₆alkyl, and —OC₁₋₆alkyl. In another class of this embodiment, each R^{a} is independently selected from the group: CN, oxo, —OH, halogen, —C₁₋₆alkyl, —C₁₋₆ alkyl-OH. —O—C₁₋₆alkyl, —C₃₋₆cycloalkyl, —C(O)C₁₋₆alkyl, —C(O)C₃₋₆cycloalkyl, —C₁₋₆alkyl-C₃₋₆cycloalkyl, —(CH₂)ₚ—O—C₁₋₆alkyl, —(CH₂)ₚ—O—C₃₋₆cycloalkyl, —(CH₂)ₚ—S(O)ᵣR^{f}, and —N(R^{g})₂, wherein each alkyl and cycloalkyl is unsubstituted or substituted with one to six substituents selected from halogen, CF₃, OH, C₁₋₆alkyl, and —OC₁₋₆alkyl. In another class of this embodiment, each R^{a} is independently selected from the group: CN, oxo, —OH, halogen, —C₁.₆alkyl, —C₁₋₆alkyl-OH, —O—C₁₋₆alkyl, —C₃₋₆cycloalkyl, —C(O)C₁₋₆alkyl, —C(O)C₃₋₆cycloalkyl, —(CH₂)ₚ—S(O)ᵣR^{f}, and —N(R^{g})₂, wherein each alkyl and cycloalkyl is unsubstituted or substituted with one to six substituents selected from halogen, CF₃, OH, C₁₋₆alkyl, and —OC₁₋₆alkyl. In another class of this embodiment, each R^{a} is independently selected from the group: CN, oxo, —OH, halogen, —C₁₋₆alkyl, —C₁₋₆alkyl-OH, —O—C₁₋₆alkyl, —C(O)C₁₋₆alkyl, —C(O)C₃₋₆cycloalkyl, —(CH₂)ₚ—S(O)ᵣR^{f}, and —N(R^{g})₂, wherein each alkyl and cycloalkyl is unsubstituted or substituted with one to six substituents selected from halogen, CF₃, OH, C₁₋₆alkyl, and —OC₁₋₆alkyl. In another class of this embodiment, each R^{a} is independently selected from the group: —OH, halogen, —C₁₋₆alkyl, —C(O)C₁₋₆alkyl, —C(O)C₃₋₆cycloalkyl, and —(CH₂)ₚ—S(O)ᵣR^{f}, wherein each alkyl and cycloalkyl is unsubstituted or substituted with one to six substituents selected from halogen, CF₃, OH. C₁₋₆alkyl, and —OC₁₋₆alkyl. In another class of this embodiment, each R^{a} is independently selected from the group: —OH, F, —CH₃, —CH₂CH₃, —C(O)CH₃, —C(O)CH₂CH₃, —C(O)cyclopropane, —C(O)cyclobutane, and —SO₂CH₃, wherein each R^{a} is unsubstituted or substituted with one to six substituents selected from halogen, CF₃, OH, C₁₋₆alkyl, and —OC₁₋₆-alkyl. In another class of this embodiment, each R^{a} is independently selected from the group: —OH, and —C₁₋₆alkyl, wherein each alkyl is unsubstituted or substituted with one to six substituents selected from halogen, CF₃, OH, C₁₋₆alkyl, and —OC₁₋₆alkyl. In another class of this embodiment, each R^{a} is independently selected from the group: —OH. —CH₃, and —CH₂CH₃, wherein —CH₃ and —CH₂CH₃ is unsubstituted or substituted with one to six substituents selected from halogen, CF₃, OH, C₁₋₆alkyl, and —OC₁₋₆alkyl.

In another embodiment, each R^{b} is independently selected from the group: CF₃, halogen, —C₁₋₆alkyl, and —C₃₋₆cycloalkyl. In a class of this embodiment, each R^{b} is independently selected from the group: CF₃, halogen, and —C₁₋₆alkyl. In another class of this embodiment, each R^{b} is independently selected from the group: CF₃, and —C₁₋₆alkyl. In another class of this embodiment, each R^{b} is CF₃, In another class of this embodiment, each R^{b} is halogen. In another class of this embodiment, each R^{b} is —C₁₋₆alkyl.

In another embodiment, each R^{c} is independently selected from the group: CN, CF₃, —OH, oxo, halogen, —C₁₋₆alkyl, —O—C₁₋₆alkyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆ cycloheteroalkyl. —(CH₂)_{q}—O—C₁₋₆alkyl, —(CH₂)_{q}—O—C₃₋₆cycloalkyl, —(CH₂)_{q}—O—C₂₋₆ cycloheteroalkyl, —(CH₂)_{q}—O-aryl, —(CH₂)_{q}—O-heteroaryl, —OC₁₋₆alkyl-C₃₋₆cycloalkyl, —OC₁₋₆-alkyl-C₂₋₆cycloheteroalkyl, —OC₁₋₆alkyl-aryl, —OC₁₋₆alkyl-heteroaryl, —(CH₂)_{q}—S(O)ᵣR^{h}, —N(Rⁱ)₂, —C(O)R^{j}, and —C(O)NRⁱ, wherein each alkyl, cycloalkyl, cycloheteroalkyl, aryl and heteroaryl is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In a class of this embodiment, each R^{c} is independently selected from the group: CN, CF₃, —OH, oxo, halogen, —C₁₋₆alkyl, —O—C₁₋₆alkyl, —C₃₋₆cycloalkyl, —C₂₋₆cycloheteroalkyl, —C₁₋₆alkyl-C₃₋₆cycloalkyl, —C₁₋₆alkyl-C₂₋₆cycloheteroalkyl, —(CH₂)_{q}—O—C₁₋₆alkyl, —(CH₂)_{q}—O—C₃₋₆cycloalkyl, —(CH₂)_{q}—O—C₂₋₆cycloheteroalkyl, —OC₁₋₆alkyl-C₃₋₆cycloalkyl, —OC₁₋₆alkyl-C₂₋₆cycloheteroalkyl, —(CH₂)_{q}—S(O)ᵣR^{h}, —N(Rⁱ)₂. —C(O)R^{j}, and —C(O)NRⁱ, wherein each alkyl, cycloalkyl, cycloheteroalkyl, aryl and heteroaryl is unsubstituted or substituted with one to six substituents selected from halogen, CF₃, CF₂H, OCF₃, CN, CH₂CF₃, CF₂CH₃, —C₁₋₆alkyl, and —OC₁₋₆-alkyl. In another class of this embodiment, each R^{c} is independently selected from the group: CN, CF₃, —OH, oxo, halogen, —C₁₋₆alkyl, —O—C₁₋₆alkyl, —(CH₂)_{q}—O—C₁₋₆alkyl, —(CH₂)_{q}—S(O)ᵣR^{h}, —N(Rⁱ)₂, —C(O)R^{j}, and —C(O)NRⁱ, wherein each alkyl is unsubstituted or substituted with one to six substituents selected from halogen, CF₃, CF₂H, OCF₃, CN. CH₂CF₃, CF₂CH₃, —C₁₋₆alkyl, and —OC₁₋₆alkyl. In another class of this embodiment, each R^{c} is independently selected from the group: CN, CF₃. —OH, oxo, halogen, —C₁₋₆alkyl, —O—C₁₋₆alkyl, and —(CH₂)_{q}—O—C₁₋₆alkyl, wherein each R^{c} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another class of this embodiment, each R^{c} is independently selected from the group: CF₃, —OH, halogen, —C₁₋₆alkyl, and —O—C₁₋₆alkyl, wherein each alkyl is unsubstituted or substituted with one to six substituents selected from halogen, CF₃, CF₂H, OCF₃, CN, CH₂CF₃, CF₂CH₃, —C₁₋₆-alkyl, and —OC₁₋₆alkyl. In another class of this embodiment, each R^{c} is independently selected from the group: CF₃, —OH, —C₁₋₆alkyl, and —O—C₁₋₆alkyl, wherein each alkyl is unsubstituted or substituted with one to six substituents selected from halogen, CF₃. CF₂H, OCF₃, CN, CH₂CF₃, CF₂CH₃, —C₁₋₆alkyl, and —OC₁₋₆alkyl. In another class of this embodiment, each R^{c} is independently selected from the group: —OH, —CH₃, —CF₃, and —OCHF₂, wherein —CH₃ is unsubstituted or substituted with one to three substituents selected from halogen, CF₃, CF₂H, OCF₃, CN. CH₂CF₃, CF₂CH₃, —C₁₋₆alkyl, and —OC₁₋₆alkyl. In another class of this embodiment, each R^{c} is independently selected from the group: —OH, CF₃, and —C₁₋₆alkyl, wherein each alkyl is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another class of this embodiment, each R^{c} is independently selected from the group: —OH, —CH₃, and —CF₃, wherein —CH₃ is unsubstituted or substituted with one to three substituents selected from halogen, CF₃, CF₂H. OCF₃, CN, CH₂CF₃, CF₂CH₃, —C₁₋₆alkyl, and —OC₁₋₆alkyl.

In another embodiment, each R^{d} is independently selected from the group: hydrogen, OH, halogen, and —C₁₋₆alkyl. In a class of this embodiment, each alkyl is independently selected from the group: hydrogen, halogen, and —C₁₋₆alkyl. In another class of this embodiment, each alkyl is independently selected from the group: hydrogen, and —C₁₋₆alkyl. In another class of this embodiment, each alkyl is independently selected from the group: hydrogen. In another class of this embodiment, each alkyl is independently selected from the group: —C₁₋₆alkyl.

In another embodiment, each R^{e} is independently selected from the group: hydrogen, OH, halogen, and —C₁₋₆alkyl. In a class of this embodiment, each R^{e} is independently selected from the group: hydrogen, halogen, and —C₁₋₆alkyl. In another class of this embodiment, each R^{e} is independently selected from the group: hydrogen, and —C₁₋₆alkyl. In another class of this embodiment, each R^{e} is independently selected from the group: hydrogen. In another class of this embodiment, each R^{e} is independently selected from the group: —C₁₋₆alkyl.

In another embodiment, each R^{f} is independently selected from the group: hydrogen, —C₁₋₆ alkyl, —C₃₋₆cycloalkyl, and —C₂₋₆cycloheteroalkyl. In a class of this embodiment, each R^{f} is independently selected from the group: hydrogen, and —C₁₋₆alkyl. In another class of this embodiment, each R^{f} is independently selected from the group: hydrogen, and CH₃. In another class of this embodiment, each R^{f} is hydrogen. In another class of this embodiment, each R^{f} is —C₁₋₆alkyl. In another class of this embodiment, each R^{f} is —CH₃,

In another embodiment, each R^{g} is independently selected from the group: hydrogen, —C₁₋₆alkyl, —C₃₋₆cycloalkyl, —C₂₋₆cycloheteroalkyl, aryl, heteroaryl, —C(O)C₁₋₆alkyl, and —S(O)ᵣR^{f}, wherein alkyl, cycloalkyl, cycloheteroalkyl, aryl and heteroaryl is unsubstituted or substituted with one to three substituents selected from: CF₃, halogen, OH and —OC₁₋₆alkyl. In a class of this embodiment, each R^{g} is independently selected from the group: hydrogen, —C₁₋₆ alkyl, —C₃₋₆cycloalkyl, —C₂₋₆cycloheteroalkyl, —C(O)C₁₋₆alkyl, and —S(O)ᵣR^{f}, wherein alkyl, cycloalkyl, and cycloheteroalkyl is unsubstituted or substituted with one to three substituents selected from: CF₃, halogen, OH and —OC₁₋₆alkyl. In another class of this embodiment, each R^{g} is independently selected from the group: hydrogen, —C₁₋₆alkyl, —C(O)C₁₋₆alkyl, and —S(O)ᵣR^{f}, wherein alkyl is unsubstituted or substituted with one to three substituents selected from: CF₃, halogen, OH and —OC₁₋₆alkyl. In another class of this embodiment, each R^{g} is independently selected from the group: hydrogen, and —C₁₋₆alkyl, wherein alkyl is unsubstituted or substituted with one to three substituents selected from: CF₃, halogen, OH and —OC₁₋₆alkyl. In another class of this embodiment, each R^{g} is hydrogen. In another class of this embodiment, each R^{g} is —C₁₋₆alkyl, wherein alkyl is unsubstituted or substituted with one to three substituents selected from: CF₃, halogen, OH and —OC₁₋₆alkyl.

In another embodiment, each R^{h} is independently selected from the group: hydrogen, —C₁₋₆alkyl, —C₃₋₆cycloalkyl, and —C₂₋₆cycloheteroalkyl. In a class of this embodiment, each R^{h} is independently selected from the group: hydrogen, and —C₁₋₆alkyl. In another class of this embodiment, each R^{h} is hydrogen. In another class of this embodiment, each R^{h} is —C₁₋₆alkyl.

In another embodiment, each Rⁱ is independently selected from the group: hydrogen, —C₁₋₆ alkyl, —C₃₋₆cycloalkyl, and —C₂₋₆cycloheteroalkyl. In a class of this embodiment, each Rⁱ is independently selected from the group: hydrogen, and —C₁₋₆alkyl. In another class of this embodiment, each Rⁱ is hydrogen. In another class of this embodiment, each Rⁱ is —C₁₋₆alkyl.

In another embodiment, each Rⁱ is independently selected from the group: OH, —C₁₋₆ alkyl, —C₃₋₆cycloalkyl, and —C₂₋₆cycloheteroalkyl, wherein alkyl, cycloalkyl, and cycloheteroalkyl is unsubstituted or substituted with one to three substituents selected from: CF₃, halogen, OH and —OC₁₋₆alkyl. In a class of this embodiment, each Rⁱ is independently selected from the group: OH, and —C₁₋₆alkyl, wherein alkyl is unsubstituted or substituted with one to three substituents selected from: CF₃, halogen, OH and —OC₁₋₆alkyl. In another class of this embodiment, each R^{j} is OH. In another class of this embodiment, each Rⁱ is —C₁₋₆alkyl, wherein alkyl is unsubstituted or substituted with one to three substituents selected from: CF₃, halogen, OH and —OC₁₋₆alkyl.

In another embodiment, each R^{k} is independently selected from the group: hydrogen, and —C₁₋₆alkyl. In a class of this embodiment, each R^{k} is hydrogen. In another class of this embodiment, each R^{k} is —C₁₋₆alkyl.

In another embodiment, p is 0, 1, 2, 3, 4, 5 or 6. In another embodiment, p is 0, 1, 2, 3, 4, or 5. In another embodiment, p is 1, 2, 3, 4, 5 or 6. In another embodiment, p is 1, 2, 3, 4 or 5. In another embodiment, p is 0, 1, 2, 3, or 4. In another embodiment, p is 1, 2, 3, or 4. In another embodiment, p is 0, 1, 2, or 3. In another embodiment, p is 1, 2, or 3. In another embodiment, p is 0, 1 or 2. In another embodiment, p is 1 or 2. In another embodiment, p is 0. In another embodiment, p is 1. In another embodiment, p is 2. In another embodiment, p is 3. In another embodiment, p is 4. In another embodiment, p is 5. In another embodiment, p is 6.

In another embodiment, q is 0, 1, 2, 3, 4, 5 or 6. In another embodiment, q is 0, 1, 2, 3, 4, or 5. In another embodiment, q is 1, 2, 3, 4, 5 or 6. In another embodiment, q is 1, 2, 3, 4 or 5. In another embodiment, q is 0, 1, 2, 3, or 4. In another embodiment, q is 1, 2, 3, or 4. In another embodiment, q is 0, 1, 2, or 3. In another embodiment, q is 1, 2, or 3. In another embodiment, q is 0, 1 or 2. In another embodiment, q is 1 or 2. In another embodiment, q is 0. In another embodiment, q is 1. In another embodiment, q is 2. In another embodiment, q is 3. In another embodiment, q is 4. In another embodiment, q is 5. In another embodiment, q is 6.

In another embodiment, r is 0, 1 or 2. In another embodiment, r is 1 or 2. In another embodiment, r is 0. In another embodiment, r is 1. In another embodiment, r is 2.

In another embodiment, the disclosure relates to compounds of structural formula Ia:

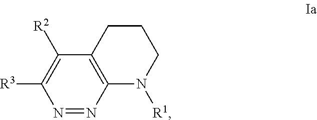

or a pharmaceutically acceptable salt thereof.

In another embodiment, the disclosure relates to compounds of structural formula Ib:

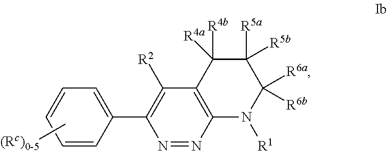

or a pharmaceutically acceptable salt thereof.

In another embodiment, the disclosure relates to compounds of structural formula Ic:

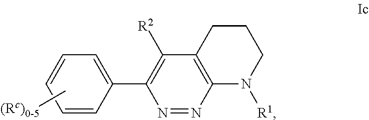

or a pharmaceutically acceptable salt thereof.

The compound of structural formula I, includes the compounds of structural formulas Ia, Ib and Ic, and pharmaceutically acceptable salts, hydrates and solvates thereof.

Another embodiment, the disclosure relates to compounds of structural formula I wherein:
R¹ is selected from the group:
1) —C₃₋₁₂cycloalkyl,
2) —C₂₋₁₁cycloheteroalkyl, and
3) —C₁₋₆alkyl-C₂₋₁₁cycloheteroalkyl,
wherein each alkyl, cycloalkyl, and cycloheteroalkyl is unsubstituted or substituted with one to six substituents selected from R^{a};
R² is selected from the group:
1) hydrogen, and
2) —C₁₋₆alkyl,
wherein alkyl is unsubstituted or substituted with one to five substituents selected from R^{b};
R³ is selected from the group:
1) aryl, and
2) heteroaryl,
wherein each aryl and heteroaryl is unsubstituted or substituted with one to five substituents selected from R^{c};
R^{4a} is selected from the group:
1) hydrogen, and
2) —C₁₋₆alkyl,
wherein each alkyl is unsubstituted or substituted with one to five substituents selected from R^{d};
R^{4b} is selected from the group:
1) hydrogen, and
2) —C₁₋₆alkyl,
wherein each alkyl is unsubstituted or substituted with one to five substituents selected from R^{d}:
R^{5a} is selected from the group:
1) hydrogen,
2) CN,
3) —C₁₋₆alkyl.
4) —O—C₁₋₆alkyl,
5) —N(R^{k})₂, and
6) halogen,
wherein each alkyl is unsubstituted or substituted with one to five substituents selected from R^{e};
R^{5b} is selected from the group:
1) hydrogen,
2) CN,
3) —C₁₋₆alkyl,
4) —O—C₁₋₆alkyl,
5) —N(R^{k})₂, and
6) halogen,
wherein each alkyl is unsubstituted or substituted with one to five substituents selected from R^{e};
R^{6a} is selected from the group:
1) hydrogen, and
2) —C₁₋₆alkyl,
wherein each alkyl is unsubstituted or substituted with one to five substituents selected from R^{e};
R^{6b} is selected from the group:
1) hydrogen, and
2) —C₁₋₆alkyl,
wherein each alkyl is unsubstituted or substituted with one to five substituents selected from R^{e}; and the other substituents are as defined above;
or a pharmaceutically acceptable salt thereof.

Another embodiment, the disclosure relates to compounds of structural formula I wherein:
R¹ is selected from the group:
1) cyclobutane,
2) cyclohexane,
3) bicyclo[3.1.1]heptane,
4) piperidine, and
5) —CH₂-pyrrolidine,
wherein R¹ is unsubstituted or substituted with one to six substituents selected from R^{a};
R² is selected from the group:
1) hydrogen, and
2) —C₁₋₆alkyl.
wherein alkyl is unsubstituted or substituted with one to five substituents selected from R^{b};
R³ is selected from the group:
1) phenyl,
2) benzofuran, and
3) pyridine,
wherein each phenyl, benzofuran and pyridine is unsubstituted or substituted with one to five substituents selected from R^{c};
R^{4a} is selected from the group:
1) hydrogen, and
2) —C₁₋₆alkyl,
wherein each alkyl is unsubstituted or substituted with one to five substituents selected from R^{d}:
R^{4b} is selected from the group:
1) hydrogen, and
2) —C₁₋₆alkyl.
wherein each alkyl is unsubstituted or substituted with one to five substituents selected from R^{d};
R^{5a} is selected from the group:
1) hydrogen,
2) CN,
3) —C₁₋₆alkyl.
4) —O—C₁₋₆alkyl,
5) —N(R^{k})₂, and
6) halogen,
wherein each alkyl is unsubstituted or substituted with one to five substituents selected from R^{e};
R^{5b} is selected from the group:
1) hydrogen,
2) CN,
3) —C₁₋₆alkyl.
4) —O—C₁₋₆alkyl,
5) —N(R^{k})₂, and
6) halogen,
wherein each alkyl is unsubstituted or substituted with one to five substituents selected from R^{e};
R^{6a} is selected from the group:
1) hydrogen, and
2) —C₁₋₆alkyl.
wherein each alkyl is unsubstituted or substituted with one to five substituents selected from R^{e};
R^{6b} is selected from the group:
1) hydrogen, and
2) —C₁₋₆alkyl,
wherein each alkyl is unsubstituted or substituted with one to five substituents selected from R^{e}; and the other substituents are as defined above;
or a pharmaceutically acceptable salt thereof.

Another embodiment, the disclosure relates to compounds of structural formula I wherein:
R¹ is selected from the group:
1) —C₃₋₁₂cycloalkyl, and
2) —C₂₋₁₁cycloheteroalkyl,
wherein each cycloalkyl and cycloheteroalkyl is unsubstituted or substituted with one to six substituents selected from R^{a};
R² is selected from the group:
1) hydrogen, and
2) —C₁₋₆alkyl.
wherein alkyl is unsubstituted or substituted with one to five substituents selected from R^{b};
R³ is aryl, wherein aryl is unsubstituted or substituted with one to five substituents selected from R^{c};
R^{4a} is selected from the group:
1) hydrogen, and
2) —C₁₋₆alkyl,
wherein each alkyl is unsubstituted or substituted with one to five substituents selected from R^{d};
R^{4b} is selected from the group:
1) hydrogen, and
2) —C₁₋₆alkyl,
wherein each alkyl is unsubstituted or substituted with one to five substituents selected from R^{d};
R^{5a} is selected from the group:
1) hydrogen, and
2) —N(R^{k})₂;
R^{5b} is selected from the group:
1) hydrogen, and
2) —N(R^{k})₂;
R^{6a} is selected from the group:
1) hydrogen, and
2) —C₁₋₆alkyl,
wherein each alkyl is unsubstituted or substituted with one to five substituents selected from R^{e}:
R^{6b} is selected from the group:
1) hydrogen, and
2) —C₁₋₆alkyl.
wherein each alkyl is unsubstituted or substituted with one to five substituents selected from R^{e}; and the other substituents are as defined above;
or a pharmaceutically acceptable salt thereof.

Another embodiment, the disclosure relates to compounds of structural formula I wherein:
R¹ is selected from the group:
1) cyclobutane,
2) cyclohexane,
3) bicyclo[3.1.1]heptane, and
4) piperidine,
wherein each cyclobutane, cyclohexane, bicyclo┌3.1.1┐heptane and piperidine is unsubstituted or substituted with one to six substituents selected from R^{a};
R² is selected from the group:
1) hydrogen, and
2) —C₁₋₆alkyl,
wherein alkyl is unsubstituted or substituted with one to five substituents selected from R^{b};
R³ is phenyl, wherein phenyl is unsubstituted or substituted with one to five substituents selected from R^{c};
R^{4a} is hydrogen;
R^{4b} is hydrogen;
R^{5a} is hydrogen.
R^{5b} is hydrogen;
R^{6a} is hydrogen; and
R^{6b} is hydrogen;
and the other substituents are as defined above;
or a pharmaceutically acceptable salt thereof.

Illustrative, but non-limiting, examples of compounds of the disclosure that are useful as inhibitors of the NLRP3 are the following compounds:
1) 2-(8-((1s,3s)-3-hydroxy-3-methylcyclobutyl)-5,6,7,8-tetrahydropyrido[2,3-c]pyridazin-3-yl)-3-methyl-5-(trifluoromethyl)phenol;
2) (R)-2-(8-(1-ethylpiperidin-3-yl)-5.6.7,8-tetrahydropyrido[2,3-c]pyridazin-3-yl)-3-methyl-5-(trifluoromethyl)phenol;
3) 5-(3-(2-hydroxy-6-methyl-4-(trifluoromethyl)phenyl)-6,7-dihydropyrido[2,3-c]pyridazin-8(5H)-yl)bicyclo[3.1.1]heptan-1-ol;
4) 2-(8-((1R,2R)-2-hydroxycyclohexyl)-5,6,7,8-tetrahydropyrido[2,3-c]pyridazin-3-yl)-3-methyl-5-(trifluoromethyl)phenol;
5) 2-(8-((1S,2R or 1R,2S)-2-hydroxy-3,3-dimethylcyclobutyl)-5,6,7,8-tetrahydropyrido[2,3-c]pyridazin-3-yl)-3-methyl-5-(trifluoromethyl)phenol:
6) 2-(8-((1R,2S or 1S,2R)-2-hydroxy-3,3-dimethylcyclobutyl)-5,6,7,8-tetrahydropyrido[2,3-c]pyridazin-3-yl)-3-methyl-5-(trifluoromethyl)phenol;
7) 2-(8-(((2S,4S)-4-fluoropyrrolidin-2-yl)methyl)-5,6,7,8-tetrahydropyrido[2,3-c]pyridazin-3-yl)-3-methyl-5-(trifluoromethyl)phenol;
8) 2-((R)-6-amino-8-((1s,3S)-3-hydroxy-3-methylcyclobutyl)-5,6,7,8-tetrahydropyrido[2,3-c]pyridazin-3-yl)-3-methyl-5-(trifluoromethyl)phenol:
9) (R)-2-(8-(1-ethylpiperidin-3-yl)-4-methyl-5,6,7,8-tetrahydropyrido[2,3-c]pyridazin-3-yl)-5-(trifluoromethyl)phenol;
10) (R)-3-(2-(difluoromethoxy)-4-(trifluoromethyl)phenyl)-8-(1-ethylpiperidin-3-yl)-4-methyl-5.6.7,8-tetrahydropyrido[2,3-c]pyridazine:
11) (R)-3-(2-(difluoromethoxy)-6-(trifluoromethyl)pyridin-3-yl)-8-(1-ethylpiperidin-3-yl)-4-methyl-5,6,7,8-tetrahy dropyrido[2,3-c]pyridazine;
12) (R)-3-(benzofuran-5-yl)-8-(1-ethylpiperidin-3-yl)-4-methyl-5,6,7,8-tetrahydropyrido[2,3-c]pyridazine;
13) 2-(8-(((2S,4S)-4-fluoro-1-methylpyrrolidin-2-yl)methyl)-5,6,7,8-tetrahydropyrido[2,3-c]pyridazin-3-yl)-3-methyl-5-(trifluoromethyl)phenol;
14) (3S,4R)-3-(3-(2-hydroxy-6-methyl-4-(trifluoromethyl)phenyl)-6,7-dihydropyrido[2,3-c]pyridazin-8(5H)-yl)-1-methylpiperidin-4-ol;
15) 2-(8-(((2S,4S)-1-ethyl-4-fluoropyrrolidin-2-yl)methyl)-5,6,7,8-tetrahydropyrido[2,3-c]pyridazin-3-yl)-3-methyl-5-(trifluoromethyl)phenol;
16) 1-((2S,4R)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)ethan-1-one;
17) 1-((2S,4S)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)ethan-1-one;
18) 1-((2S,4R)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)propan-1-one;
19) cyclopropyl((2S,4R)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)methanone;
20) cyclobutyl((2S,4R)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)methanone:
21) 2-(8-(((2S,4R)-4-fluoro-1-(methylsulfonyl)pyrrolidin-2-yl)methyl)-5,6,7,8-tetrahydropyrido[2,3-c]pyridazin-3-yl)-3-methyl-5-(trifluoromethyl)phenol; and
22) 2-(8-(((2S,4S)-4-fluoro-1-(methylsulfonyl)pyrrolidin-2-yl)methyl)-5,6,7,8-tetrahydropyrido[2,3-c]pyridazin-3-yl)-3-methyl-5-(trifluoromethyl)phenol;
or pharmaceutically acceptable salts thereof.

Although the specific stereochemistries described above are preferred, other stereoisomers, including diastereoisomers, enantiomers, epimers, and mixtures of these may also have utility in treating NLRP3 mediated diseases.

Synthetic methods for making the compounds are disclosed in the Examples shown below. Where synthetic details are not provided in the examples, the compounds are readily made by a person of ordinary skill in the art of medicinal chemistry or synthetic organic chemistry by applying the synthetic information provided herein. Where a stereochemical center is not defined, the structure represents a mixture of stereoisomers at that center. For such compounds, the individual stereoisomers, including enantiomers, diastereoisomers, and mixtures of these are also compounds of the disclosure.

### Definitions

“Ac” is acetyl, which is CH₃C(=O)—.

“Alkyl” means saturated carbon chains which may be linear or branched or combinations thereof, unless the carbon chain is defined otherwise. Other groups having the prefix, alk”, such as alkoxy and alkanoyl, also may be linear or branched, or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched, or combinations thereof, unless otherwise defined. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched, or combinations thereof, unless otherwise defined. Examples of alkynyl include ethynyl, propargyl, 3-methyl-1-pentynyl, 2-heptynyl and the like.

“Cycloalkyl” means a saturated monocyclic, bicyclic, spirocyclic, fused or bridged carbocyclic ring, having a specified number of carbon atoms. Examples of cycloalkyl include cyclopropyl, cyclobutyl, cyclopentyl, cyclohexyl, cycloheptyl, and the like. In one embodiment, cycloalkyl is-C₃₋₁₂cycloalkyl. In another embodiment, cycloalkyl is selected from: cyclopropyane, and cyclobutane. In another embodiment, cycloalkyl is selected from: cyclobutane, cyclohexane, and bicyclo[3.1.1]heptane.

“Cycloalkenyl” means a monocyclic, bicyclic, spirocyclic, fused or bridged carbocyclic ring, having a specified number of carbon atoms with at least one double bond. Examples of cycloalkenyl include cyclopropene, cyclobutane, cyclopentene, cyclohexene, cycloheptene, and the like. In one embodiment, cycloalkenyl is —C₃₋₁₂cycloalkenvl.

“Cycloheteroalkyl” means a monocyclic, bicyclic, spirocyclic, fused or bridged ring or ring system having a specified number of carbon atoms and containing at least one saturated ring wherein at least one ring heteroatom selected from N. NH, S (including SO and SO₂) and O, or with at least one partially unsaturated ring wherein at least one ring heteroatom selected from N, NH, S (including SO and SO₂) and O. The cycloheteroalkyl ring may be substituted on the ring carbons and/or the ring nitrogen or sulfur. The cycloheteroalkyl ring may be fused to an aryl or heteroaryl ring. Examples of cycloheteroalkyl include tetrahydrofuranyl, pyrrolidinyl, tetrahydrothiophenyl, azetidinyl, piperazinyl, piperidinyl, morpholinyl, oxetanyl and tetrahydropyranyl. In one embodiment, cycloheteroalkyl is C₂₋₁₁cycloheteroalkyl. In another embodiment, cycloheteroalkyl is piperidine. In another embodiment, cycloheteroalkyl is pyrrolidine.

“Cycloheteroalkenyl” means a monocyclic, bicyclic, spirocyclic, fused, or bridged ring or ring system having a specified number of carbon atoms and containing at least one double bond and at least one heteroatom selected from N, NH. S (including SO and SO₂) and O. Examples of cycloheteroalkenyl include dihydropyran and dihydrofuran, and the like.

“Aryl” means a monocyclic, bicyclic or tricyclic carbocyclic aromatic ring or ring system containing 6-14 carbon atoms, wherein at least one of the rings is aromatic. Examples of aryl include phenyl and naphthyl. In one embodiment, aryl is phenyl.

“Heteroaryl” means a monocyclic, bicyclic or tricyclic ring or ring system containing 5-14 ring atoms and containing at least one ring heteroatom selected from N, NH, S (including SO and SO₂) and O, wherein at least one of the heteroatom containing rings is aromatic. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzofuranyl, benzothiophenyl, quinolyl, indolyl, isoquinolyl, quinazolinyl, dibenzofuranyl, and the like. In one embodiment, heteroaryl is pyridine or benzofuran. In another embodiment, heteroaryl is pyridine. In another embodiment, heteroaryl is benzofuran.

“Halogen” includes fluorine, chlorine, bromine and iodine. In one embodiment, halogen is fluorine, chorine or bromine. In another embodiment, halogen is fluorine or chlorine. In another embodiment, halogen is chlorine or bromine. In another embodiment, halogen is fluorine or bromine. In another embodiment, halogen is fluorine. In another embodiment, halogen is chlorine. In another embodiment, halogen is bromine.

“Me” represents methyl.

“Oxo” represents═O.

“Saturated” means containing only single bonds.

“Unsaturated” means containing at least one double or triple bond. In one embodiment, unsaturated means containing at least one double bond. In another embodiment, unsaturated means containing at least one triple bond.

When any variable (e.g., R¹, R^{a}, etc.) occurs more than one time in any constituent or in structural formula I, its definition on each occurrence is independent of its definition at every other occurrence. Also, combinations of substituents and/or variables are permissible only if such combinations result in stable compounds. A squiggly line across a bond in a substituent variable represents the point of attachment.

Under nomenclature used throughout this disclosure, the point of attachment is described first, followed by the terminal portion of the designated side chain. For example, a C₁₋₅ alkylcarbonylamino C₁₋₆ alkyl substituent is equivalent to:

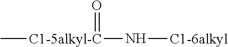

In choosing compounds of the present disclosure, one of ordinary skill in the art will recognize that the various substituents, i.e., R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The phrase “pharmaceutically acceptable” is employed herein to refer to those compounds, materials, compositions, salts and/or dosage forms which are, using sound medical judgment, and following all applicable government regulations, safe and suitable for administration to a human being or an animal.

Compounds of structural formula I may contain one or more asymmetric centers and can thus occur as racemates and racemic mixtures, single enantiomers, diastereomeric mixtures and individual diastereomers. The present disclosure is meant to encompass all such isomeric forms of the compounds of structural formula I.

The independent syntheses of optical isomers and diastereoisomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration or sufficient heavy atoms to make an absolute assignment.

If desired, racemic mixtures of the compounds may be separated so that the individual enantiomers are isolated. The separation can be carried out by methods well-known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of structural formula I.

In the compounds of structural formula I, the atoms may exhibit their natural isotopic abundances, or one or more of the atoms may be artificially enriched in a particular isotope having the same atomic number, but an atomic mass or mass number different from the atomic mass or mass number predominately found in nature. The present disclosure is meant to include all suitable isotopic variations of the compounds of structural formula I. For example, different isotopic forms of hydrogen (H) include protium (¹H), deuterium (²H), and tritium (³H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Tritium is radioactive and may therefore provide for a radiolabeled compound, useful as a tracer in metabolic or kinetic studies. Isotopically-enriched compounds within structural formula I, can be prepared without undue experimentation by conventional techniques well known to those skilled in the art or by processes analogous to those described in the Schemes and Examples herein using appropriate isotopically-enriched reagents and/or intermediates.

Furthermore, some of the crystalline forms for compounds of the present disclosure may exist as polymorphs and as such are intended to be included in the present disclosure. In addition, some of the compounds of the present disclosure may form solvates with water or common organic solvents. Such solvates are encompassed within the scope of this disclosure.

It is generally preferable to administer compounds of the present disclosure as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

### Salts

It will be understood that, as used herein, references to the compounds of the present disclosure are meant to also include the pharmaceutically acceptable salts, and also salts that are not pharmaceutically acceptable when they are used as precursors to the free compounds or their pharmaceutically acceptable salts or in other synthetic manipulations.

The compounds of the present disclosure may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this disclosure which are generally prepared by reacting the free base with a suitable organic or inorganic acid. Representative salts of basic compounds of the present disclosure include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, formic, fumarate, gluceptate, gluconate, glutamate, glycollylars-anilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, tartrate, teoclate, tosylate, triethiodide, trifluoroacetate and valerate. Where the compounds of the disclosure carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present disclosure, pharmaceutically acceptable esters of carboxylic acid derivatives, such as methyl, ethyl, or pivaloyloxymethyl, or acyl derivatives of alcohols, such as O-acetyl. O-pivaloyl, O-benzoyl, and O-aminoacyl, can be employed. Included are those esters and acyl groups known in the art for modifying the solubility or hydrolysis characteristics for use as sustained-release or prodrug formulations.

The term “prodrug” means compounds that are rapidly transformed, for example, by hydrolysis in blood, in vivo to the parent compound, e.g., conversion of a prodrug of structural formula I to a compound of structural formula I, or to a salt thereof: a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press. 1987, both of which are incorporated herein by reference. This disclosure includes prodrugs of the compounds of structural formula I. Solvates, and in particular, the hydrates of the compounds of structural formula I are also included in the present disclosure as well.

### Utilities

The compounds of structural formula I are potent inhibitors of Nod-Like Receptor Protein 3 (NLPR3). The compounds of structural formula I, and pharmaceutically acceptable salts thereof, may be efficacious in the treatment of diseases, disorders and conditions that are mediated by the inhibition of Nod-Like Receptor Protein 3 (NLPR3).

The present disclosure relates to the treatment or prevention of a disease, disorder or condition mediated by NLRP3 such as inflammation, an auto-immune disease, a cancer, an infection, a disease or disorder of the central nervous system, a metabolic disease, a cardiovascular disease, a fibrotic disease or fibrosis, a respiratory disease, a kidney disease, a liver disease, an ophthalmic or ocular disease, a skin disease, a lymphatic disease, a rheumatic disease, graft versus host disease, allodynia, or an NLRP3-related disease in a subject that has been determined to carry a germline or somatic non-silent mutation in NLRP3.

The disease, disorder or condition mediated by NLRP3 includes but is not limited to, gout, pseudogout, osteoarthritis, familial cold autoinflammatory syndrome. Muckle-Wells syndrome, neonatal onset multisystem inflammatory disease, diabetes, NASH, sepsis, age related macular degeneration, diabetic retinopathy, liver fibrosis, kidney fibrosis, atherosclerosis, heart failure, peripheral artery disease, myeloproliferative neoplasm, leukemia, myelodysplastic syndrome, myelofibrosis, lung cancer, colon cancer, Parkinson's disease. Alzheimer's disease, traumatic brain injury, spinal cord injury, amyotrophic lateral sclerosis, multiple sclerosis, atopic dermatitis, hidradenitis suppurativa, pericarditis, myocarditis, preeclampsia, dermatomyositis, Still's disease, juvenile idiopathic arthritis, age related macular degeneration, diabetic retinopathy, acute kidney disease, a chronic kidney disease, or a rare kidney disease. Diseases, disorders or conditions mediated by Nod-Like Receptor Protein 3 (NLPR3)), also include, but are not limited to, gout, pseudogout. CAPS, NASH, fibrosis, osteoarthritis, atherosclerosis, heart failure, idiophathic pericarditis, myocarditis, atopic dermatitis, hidradenitis suppurativa, inflammatory bowel disease, cancer, Alzheimer's Disease, Parkinson's Disease and traumatic brain injury.

In one embodiment, the condition, disease or disorder is an inflammatory joint disease such as gout, pseudogout, or osteoarthritis.

In another embodiment, the cryopyrin-associated autoinflammatory syndrome is familial cold autoinflammatory syndrome, Muckle-Wells syndrome, or neonatal onset multisystem inflammatory disease.

In another embodiment, the metabolic disease is diabetes.

In another embodiment, the liver disease is NASH.

In another embodiment, the infection is sepsis.

In another embodiment, the ophthalmic or ocular disease is age related macular degeneration or diabetic retinopathy.

In another embodiment, the fibrotic disease is liver fibrosis or kidney fibrosis.

In some embodiments, the cardiovascular disease is atherosclerosis, heart failure or peripheral artery disease.

In another embodiment, the cancer is myeloproliferative neoplasm, leukemia, myelodysplastic syndrome, myelofibrosis, lung cancer or colon cancer.

In another embodiment, the condition, disease or disorder of the central nervous system is Parkinson's disease, Alzheimer's disease, traumatic brain injury, spinal cord injury, amyotrophic lateral sclerosis, or multiple sclerosis.

In another embodiment, the skin disease is atopic dermatitis or hidradenitis suppurativa (HS).

In another embodiment, the inflammatory disease is pericarditis or myocarditis.

In another embodiment, the inflammatory disease is preeclampsia.

In another embodiment, the rheumatic disease is dermatomyositis, Still's disease, or juvenile idiopathic arthritis.

In another embodiment, the ocular disease is age related macular degeneration, or diabetic retinopathy.

In another embodiment, the kidney disease is an acute kidney disease, a chronic kidney disease, or a rare kidney disease.

One or more of these conditions or diseases may be treated, managed, prevented, reduced, alleviated, ameliorated or controlled by the administration of a therapeutically effective amount of a compound of structural formula I. or a pharmaceutically acceptable salt thereof, to a patient in need of treatment.

The compounds of structural formula I may also be used for the manufacture of a medicament which may be useful for treating, preventing, managing, alleviating, ameliorating or controlling one or more of these conditions, diseases or disorders, including but not limited to: gout, pseudogout, osteoarthritis, familial cold autoinflammatory syndrome, Muckle-Wells syndrome, neonatal onset multisystem inflammatory disease, diabetes, NASH, sepsis, age related macular degeneration, diabetic retinopathy, liver fibrosis, kidney fibrosis, atherosclerosis, heart failure, peripheral artery disease, myeloproliferative neoplasm, leukemia, myelodysplastic syndrome, myelofibrosis, lung cancer, colon cancer, Parkinson's disease. Alzheimer's disease, traumatic brain injury, spinal cord injury, amyotrophic lateral sclerosis, multiple sclerosis, atopic dermatitis, hidradenitis suppurativa, pericarditis, myocarditis, preeclampsia, dermatomyositis, Still's disease, juvenile idiopathic arthritis, age related macular degeneration, diabetic retinopathy, acute kidney disease, a chronic kidney disease, or a rare kidney disease. The compounds of structural formula I may also be used for the manufacture of a medicament which may be useful for treating, preventing, managing, alleviating, ameliorating or controlling one or more of these conditions, diseases or disorders, including but not limited to: gout, pseudogout, CAPS, NASH, fibrosis, osteoarthritis, atherosclerosis, heart failure, idiophathic pericarditis, myocarditis, atopic dermatitis, hidradenitis suppurativa, inflammatory bowel disease, cancer, Alzheimer's Disease, Parkinson's Disease and traumatic brain injury.

Preferred uses of the compounds may be for the treatment of one or more of the following diseases by administering a therapeutically effective amount to a patient in need of treatment.

The compounds may be used for manufacturing a medicament for the treatment of one or more of these diseases:
1) gout,
2) pseudogout,
3) cryopyrin-associated periodic syndromes.
4) non-alcoholic steatohepatitis,
5) fibrosis,
6) osteoarthritis,
7) atherosclerosis,
8) atopic dermatitis,
9) hidradenitis suppurativa,
10) Alzheimer's Disease, and
11) Parkinson's Disease.

Treatment of a disease, disorder or condition mediated by NLPR3 or the NLPR3 inflammasome pathway refers to the administration of the compounds of structural formula I to a subject with the disease, disorder or condition.

One outcome of treatment may be reducing the disease, disorder or condition mediated by NLPR3 or the NLPR3 inflammasome pathway. Another outcome of treatment may be alleviating the disease, disorder or condition mediated by NLPR3 or the NLPR3 inflammasome pathway. Another outcome of treatment may be ameliorating the disease, disorder or condition mediated by NLPR3 or the NLPR3 inflammasome pathway. Another outcome of treatment may be suppressing the disease, disorder or condition mediated by mediated by NLPR3 or the NLPR3 inflammasome pathway. Another outcome of treatment may be managing the disease, disorder or condition mediated by NLPR3 or the NLPR3 inflammasome pathway. Another outcome of treatment may be preventing the disease, disorder or condition mediated by NLPR3 or the NLPR3 inflammasome pathway.

Prevention of the disease, disorder or condition mediated by NLPR3 or the NLPR3 inflammasome pathway refers to the administration of the compounds of structural formula I to a subject at risk of the disease, disorder or condition. One outcome of prevention may be reducing the disease, disorder or condition mediated by NLPR3 or the NLPR3 inflammasome pathway in a subject at risk of the disease, disorder or condition. Another outcome of prevention may be suppressing the disease, disorder or condition mediated by NLPR3 or the NLPR3 inflammasome pathway in a subject at risk of the disease, disorder or condition. Another outcome of prevention may be ameliorating the disease, disorder or condition mediated by NLPR3 or the NLPR3 inflammasome pathway in a subject at risk of the disease, disorder or condition. Another outcome of prevention may be alleviating the disease, disorder or condition mediated by NLPR3 or the NLPR3 inflammasome pathway in a subject at risk of the disease, disorder or condition. Another outcome of prevention may be managing the disease, disorder or condition mediated by NLPR3 or the NLPR3 inflammasome pathway in a subject at risk of the disease, disorder or condition.

The terms “administration of” and or “administering a” compound should be understood to mean providing a compound of structural formula I or a prodrug of a compound of structural formula I to the individual or mammal in need of treatment.

The administration of the compound of structural formula I in order to practice the present methods of therapy is carried out by administering an effective amount of the compound of structural formula I to the mammal in need of such treatment or prophylaxis. The need for a prophylactic administration according to the methods of the present disclosure is determined via the use of well known risk factors. The effective amount of an individual compound is determined, in the final analysis, by the physician or veterinarian in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The usefulness of the present compounds in these diseases or disorders may be demonstrated in animal disease models that have been reported in the literature.

### Administration and Dose Ranges

Any suitable route of administration may be employed for providing a mammal, especially a human, with an effective dose of a compound of structural formula I. For example, oral, intravenous, infusion, subcutaneous, transcutaneous, intramuscular, intradermal, transmucosal, intramucosal, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of structural formula I are administered orally.

In the treatment or prevention of disorders, diseases and/or conditions which require inhibition of NLRP3 a suitable dosage level will generally be about 0.0001 to 500 mg per kg patient body weight per day which can be administered in single or multiple doses. In one embodiment, a suitable dosage level may be about 0.001 to 500 mg per kg patient body weight per day. In another embodiment, a suitable dosage level may be about 0.001 to about 250 mg/kg per day. In another embodiment, a suitable dosage level may be about 0.01 to about 250 mg/kg per day. In another embodiment, a suitable dosage level may be about 0.1 to about 100 mg/kg per day. In another embodiment, a suitable dosage level may be about 0.05 to 100 mg/kg per day. In another embodiment, a suitable dosage level may be about 0.1 to 50 mg/kg per day. In another embodiment, a suitable dosage level may be about 0.05 to 0.5 mg/kg per day. In another embodiment, a suitable dosage level may be about 0.5 to 5 mg/kg per day. In another embodiment, a suitable dosage level may be about 5 to 50 mg/kg per day. For oral administration, the compositions are preferably provided in the form of tablets containing 0.01 to 1000 mg of the active ingredient, particularly 0.01, 0.025, 0.05, 0.075, 0.1, 0.25, 0.5, 0.75, 1.0, 2.5, 5.0, 7.5, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 8 times per day; preferably, 1 to 4 times a day; more preferably once or twice per day, even more preferably once a day. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of structural formula I may be used in pharmaceutical compositions comprising (a) the compound(s) or pharmaceutically acceptable salts thereof, and (b) a pharmaceutically acceptable carrier. The compounds of structural formula I may be used in pharmaceutical compositions in which the compound of structural formula I or a pharmaceutically acceptable salt thereof is the only active ingredient. The compounds of structural formula I may also be used in pharmaceutical compositions that include one or more other active pharmaceutical ingredients.

The term “composition,” as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of structural formula I and a pharmaceutically acceptable carrier.

Compounds of structural formula I may be used in combination with other drugs that may also be useful in the treatment or amelioration of the diseases or conditions for which compounds of structural formula I are useful. Such other drugs may be administered, by a route and in an amount commonly used therefore, contemporaneously or sequentially with a compound of structural formula I. In the treatment of patients who suffer from chronic inflammatory conditions, more than one drug may be administered. The compounds of structural formula I may generally be administered to a patient who is already taking one or more other drugs for these conditions. Often the compounds will be administered to a patient who is already being treated with one or more anti-pain compounds when the patient's pain is not adequately responding to treatment.

The combination therapy also includes therapies in which the compound of structural formula I and one or more other drugs are administered on different overlapping schedules. It is also contemplated that when used in combination with one or more other active ingredients, the compound of structural formula I and the other active ingredients may be used in lower doses than when each is used singly. Accordingly, the pharmaceutical compositions of the present disclosure include those that contain one or more other active ingredients, in addition to a compound of structural formula I.

Examples of other active ingredients that may be administered in combination with a compound of structural formula I, and either administered separately or in the same pharmaceutical composition, include but are not limited to:
(i) anti-steatotic agents;
(ii) anti-inflammatory agents;
(iii) immunooncology agent;
(iv) lipid-lowering agents;
(v) cholesterol lowering agents;
(vi) glucose-lowering agents, including SGLT2 inhibitors;
(vii) anti-neovascular agents;
(viii) nonsteroidal anti-inflammatory drugs (“NSAIDs”):
(ix) acetyl-salicylic acid drugs (ASA) including aspirin; paracetamol;
(x) regenerative therapy treatments;
(xi) checkpoint inhibitors including anti-PD1 and anti-PDL1 inhibitors;
(xii) chemotherapy procedures;
(xiii) radiation therapy;
(xiv) surgical procedures;
(xv) urate-lowering therapy;
(xvi) anabolics and cartilage regenerative therapy;
(xvii) anti-fibrotics;
(xviii) JAK inhibitors:
(xix) TNF-alpha inhibitors:
(xx) anti-hypertensive agents; and
(xxi) STING/cGAS antagonists pharmaceutically acceptable salts thereof.

In another embodiment, the pharmaceutical composition comprises:
1) a compound of Claim 1, or a pharmaceutically acceptable salt thereof:
2) one or more compounds, or pharmaceutically acceptable salts thereof, selected from the group:
(i) anti-steatotic agents;
(ii) anti-inflammatory agents:
(iii) immunooncology agent;
(iv) lipid-lowering agents;
(v) cholesterol lowering agents;
(vi) glucose-lowering agents, including SGLT2 inhibitors;
(vii) anti-neovascular agents;
(viii) nonsteroidal anti-inflammatory drugs (“NSAIDs”);
(ix) acetyl-salicylic acid drugs (ASA) including aspirin; paracetamol;
(x) regenerative therapy treatments;
(xi) checkpoint inhibitors including anti-PD1 and anti-PDL1 inhibitors;
(xii) chemotherapy procedures;
(xiii) radiation therapy:
(xiv) surgical procedures;
(xv) urate-lowering therapy;
(xvi) anabolics and cartilage regenerative therapy;
(xvii) anti-fibrotics;
(xviii) JAK inhibitors:
(xix) TNF-alpha inhibitors;
(xx) anti-hypertensive agents; and
(xxi) STING/cGAS antagonists; and
   pharmaceutically acceptable salts thereof; and
3) a pharmaceutically acceptable carrier.

Specific compounds of use in combination with a compound of structural formula I include: anti-steatotic agents, including but not limited to. DGAT2 inhibitors.

Suitable anti-inflammatory agents include, but are not limited to, TNFα inhibitors, JAK inhibitors and NSAIDs.

Suitable lipid-lowering agents include, but are not limited to statins and PCSK9.

Suitable immunooncology agents include, but are not limited to, PD-L1 inhibitors and PD-1 inhibitors and STING antagonists.

Suitable glucose-lowering agents include, but are not limited to, insulin, SGLT2 inhibitors, metformin, GLP1-agonists.

Suitable anti-neovascular agents include, but are not limited to, anti-VEG-F treatment.

Suitable NSAIDs or non-steroidal anti-inflammatory drugs include, but are not limited to, aspirin, diclofenac, diflunisal, etodolac, fenoprofin, flurbiprofen, ibuprofen, indomethacin, ketoprofen, meclofenamic acid, mefenamic acid, meloxicam, naproxen, naproxen sodium, oxaprozin, piroxicam, sulindac, and tolmetin.

Suitable analgesics include, but are not limited to, acetaminophen and duloxetine.

The above combinations include combinations of a compound of structural formula I not only with one other active compound, but also with two or more other active compounds. Non-limiting examples include combinations of compounds with two or more active compounds selected from: anti-steatotic agents, anti-inflammatory agents, lipid-lowering agents, anti-fibrosis, immunooncology agents, glucose-lowering agents and anti-neovascular agents, NSAIDs (non-steroidal anti-inflammatory drugs), and an analgesics.

The present disclosure also provides a method for the treatment or prevention of a NLRP3 mediated disease, disorder or condition, which method comprises administration to a patient in need of such treatment or at risk of developing a NLRP3 mediated disease with a therapeutically effective amount of a NLRP3 inhibitor and an amount of one or more active ingredients, such that together they give effective relief.

In a further aspect of the present disclosure, there is provided a pharmaceutical composition comprising a NLRP3 inhibitor and one or more active ingredients, together with at least one pharmaceutically acceptable carrier or excipient.

Thus, according to a further aspect of the present disclosure there is provided the use of a NLRP3 inhibitor and one or more active ingredients for the manufacture of a medicament for the treatment or prevention of an NLRP3-mediated disease, disorder or condition. In a further or alternative aspect of the present disclosure, there is therefore provided a product comprising a NLRP3 inhibitor and one or more active ingredients as a combined preparation for simultaneous, separate or sequential use in the treatment or prevention of an NLRP3-mediated disease, disorder or condition. Such a combined preparation may be, for example, in the form of a twin pack.

It will be appreciated that for the treatment or prevention of cardiometabolic disease, neurodegenerative disease and inflammatory joint diseases, fibrosis, cancer, a compound of structural formula I may be used in conjunction with another pharmaceutical agent effective to treat that disease, disorder or conditon.

The present disclosure also provides a method for the treatment or prevention of chronic inflammatory conditions, which method comprises administration to a patient in need of such treatment an amount of a compound of structural formula I and an amount of another pharmaceutical agent effective to threat that disorder, disease or condition, such that together they give effective relief.

The present disclosure also provides a method for the treatment or prevention of chronic inflammatory conditions, which method comprises administration to a patient in need of such treatment an amount of a compound of structural formula I and an amount of another pharmaceutical agent useful in treating that particular condition, disorder or disease, such that together they give effective relief.

The term “therapeutically effective amount” means the amount the compound of structural formula I that will elicit the biological or medical response of a cell, tissue, system, animal or human that is being sought by the researcher, veterinarian, medical doctor or other clinician, which includes alleviation of the symptoms of the disorder being treated. The novel methods of treatment of this disclosure are for disorders known to those skilled in the art. The term “mammal” includes humans, and companion animals such as dogs and cats.

The weight ratio of the compound of structural formula I to the second active ingredient may be varied and will depend upon the effective dose of each ingredient. Generally, an effective dose of each will be used. Thus, for example, when a compound of structural formula I is combined with an anti-steatotic agent, the weight ratio of the compound of structural formula I generally range from about 1000:1 to about 1:1000, preferably about 200:1 to about 1:200. Combinations of a compound of structural formula I and other active ingredients will generally also be within the aforementioned range, but in each case, an effective dose of each active ingredient should be used.

### Methods of Synthesis

The following reaction schemes and Examples illustrate methods which may be employed for the synthesis of the compounds of structural formula I described in this disclosure. These reaction schemes and Examples are provided to illustrate the disclosure and are not to be construed as limiting the disclosure in any manner. All substituents are as defined above unless indicated otherwise. Several strategies based upon synthetic transformations known in the literature of organic synthesis may be employed for the preparation of the compounds of structural formula I. The scope of the disclosure is defined by the appended claims. Compound names were generated in Chemdraw Version 21.0.0.28.

### Instrumentation

Reverse phase chromatography was carried out on a Waters 150 equipped with a column selected from the following: Phenomenex Synergi C₁₈ (250 mm×30 mm×4 micron), Phenomenex Luna C₁₈ (250 mm×21 mm×5 micron), Agilent Zorbax Bonus-RP (150 mm×21 mm×5 micron), Waters X-Select CSH C₁₈ (150 mm×19 mm×5 micron). Conditions included either high pH (0-100% acetonitrile/water eluent comprising 0.1% v/v NH40H) or low pH (0-100% acetonitrile/water eluent comprising 0.1% v/v TFA or Formic Acid) and are noted for some examples. SFC chiral resolution was carried out on Waters Thar 80 SFC or Berger M G II preparative SFC systems using the following conditions: Chiral Method A: ColumnTek Enantiocel A2S-5 column, 20% EtOH (0.1% Et₂NH)/CO₂.

LC/MS determinations were carried out on Waters ACQUITY UPLC equipped with a DAD and QDa MS detectors using the following conditions: Waters ACQUITY UPLC BEH C_{18 1.7} mm 2.1×50 mm column using mobile phase containing A: 0.1% TFA in water and B: 0.1% TFA in acetonitrile with a gradient from 10% B to 90% B over 2.0 min and hold at 90% B for 0.4 min at a flow rate of 0.5 mL/min. Proton or ¹H NMR was acquired using a Bruker 500 MHz NEO NMR spectrometer equipped with a 5 mm iProbe in accordance with standard analytical techniques, unless specified otherwise, and results of spectral analysis are reported. Chemical shift (6) values are reported in delta (6) units, parts per million (ppm). Chemical shifts for ¹H NMR spectra are given relative to signals for residual non-deuterated solvent (CDCl₃ referenced at b 7.26 ppm; DMSO d-6 referenced at δ 2.50 ppm and CD₃OD referenced at δ 3.31 ppm). Multiples are reported by the following abbreviations: s=singlet, d=doublet, t=triplet, q=quartet, dd=doublet of doublets, m=multiplet or overlap ofnonequivalent resonances. Coupling constants (J) are reported in Hertz (Hz).

### Abbreviations

“*” in a molecule designates a stereocenter; Ac is acetyl; OAc is acetate; AcOH is acetic acid; aq. is aqueous; B₂pin₂ is bis(pinacolato)diboron; BPin ester is boronic acid pinacol ester; Boc or boc is tert-butoxycarbonyl; br is broad; ° C. is degrees Celsius; calc'd is calculated; cat, is catalytic; 6 is chemical shift: d is doublet; DCM is dichloromethane; dd is doublet of doublets: DIPEA is N,N-diisopropylethylamine; DMA is dimethylacetamide; DMF is dimethylformamide; DMSO is dimethylsulfoxide; DMSO-d₆ is deuterated dimethylsulfoxide; dppf is 1,1′-bis(diphenyl-phosphino)-ferrocene; Et is ethyl; Et₃N is triethylamine; EtOAc is ethyl acetate; EtOH is ethanol; FA is formic acid; g is grams; h is hour(s); HATU is 1-[Bis(dimethylamino)-methylene]-1H-1,2,3-triazolo[4,5-b]pyridinium 3-oxide hexafluorophosphate; HPLC is high-performance liquid chromatography; Hz is hertz; ⁱPr is isopropyl; J is coupling constant; kg is kilogram(s): L is liter; LC is liquid chromatography; LCMS is liquid chromatography/mass spectrometry; m is multiplet; M is molar; Me is methyl: MeCN is acetonitrile; MeOD-d₄ is deutated methanol; MeOH is methanol; mg is milligrams: MHz is megahertz; mim is minutes; mL is milliliter; mM is millimolar; mmol is millimole(s); μL is microliter: MS is mass spectrometry; MsCl is methanesulfonyl chloride; nM is nanomolar; NMP is N-methylpyrrolidone: NMR is Nuclear Magnetic Resonance; PCy₃ Pd G3 is [(Tricyclohexyl-phosphine)-2-(2′-aminobiphenyl)]-palladium(II) methanesulfonate; Pd(dppf)C₁₂ is [1,1′-bis-(diphenylphosphino)-ferrocene]-dichloropalladium(II): PG is protecting group; PIDA is phenyl-iodine(ILI) diacetate; q is quartet: rac is racemic mixture; s is singlet; sat. is saturated; SFC is Supercritical Fluid Chromatography; S_{N}Ar is nucleophilic aromatic substitution; t is triplet; ^{t}Bu is tert-butyl; tert is tertiary; TEMPO is (2,2,6,6-tetramethylpiperidin-1-yl)oxyl or (2,2,6,6-tetramethylpiperidin-1-yl)oxidanyl; TFA is trifluoroacetic acid; THF is tetrahydrofuran: TLC is thin layer chromatography; tt is triplet of triplets; XPhos Pd G3 is (2-Dicyclohexylphosphino-2′,4′,6′-triisopropyl-1,1′-biphenyl)[2-(2′-amino-1,1′-biphenyl)]palladium(II) methanesulfonate; and UV is ultraviolet.

### Chiral Separation Methods

The general preparative conditions of separating diastereomeric or enantiomeric mixtures of compounds using chiral SFC are as follows:

| Chiral Column | Stationary Phase | Method |
|---|---|---|
| ColumnTek Enantiocel A2S-5 | 20% EtOH (0.1% Et2NH)/CO₂ | A |

### General Schemes

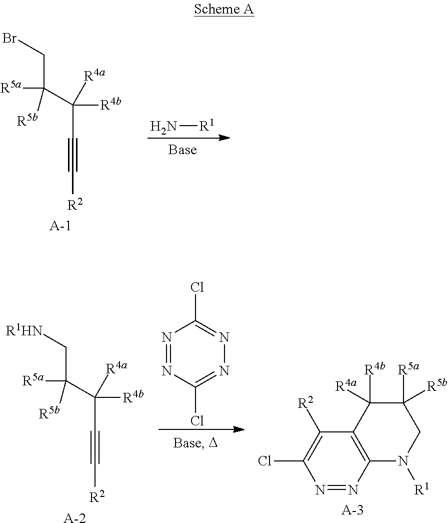

Scheme A illustrates the synthetic sequence for the preparation of pyrazolopiperidine cores of the formula A-3. Various primary amines are alkylated with bromopentynes of the formula A-1 to generate aminoalkynes of the formula A-2. Reaction of aminoalkynes A-2 with dichlorotetrazine in the presence of a base (e.g. Et₃N) and elevated temperature affords pyridazopyrrolidines of the formula A-3 via a sequence of S_{N}Ar, hetero Diels-Alder cycloaddition, and retro Diels-Alder.

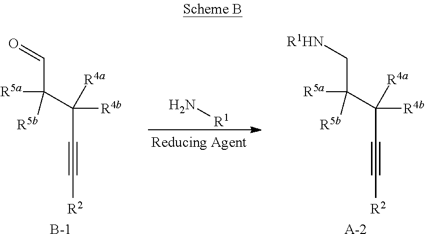

Scheme B illustrates an alternative preparation of aminoalkynes of the formula A-2. Alkynyl aldehydes of the formula B-1, either isolated or generated in situ are treated with various primary amines in the presence of a reducing agent (e.g. sodium triacetoxyborohydride or sodium cyanoborohydride) to provide the desired aminoalkynes. These can be taken forward using the cycloaddition detailed in Scheme A.

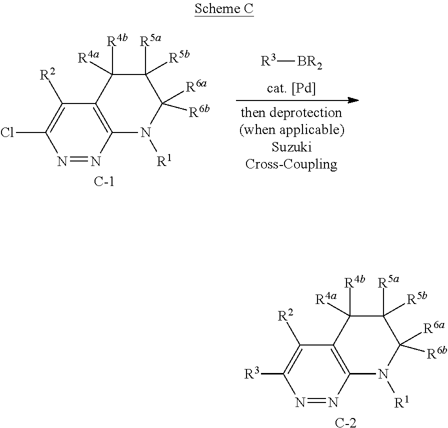

Scheme C illustrates the synthetic sequence for the preparation of biaryl pyridazine derivatives of the formula C-2. Cross-coupling of chloropyridazines of the formula C-1 using an appropriate aryl nucleophile (e.g. aryl boronic acid) and palladium catalyst gives biaryl products which are then deprotected (when applicable) to afford compounds of the formula C-2.

### Intermediate 1

### tert-butyl (3S,4R)-3-(3-chloro-6,7-dihydropyrido[2,3-c]pyridazin-8(5H)-yl)-4-hydroxypiperidine-1-carboxylate

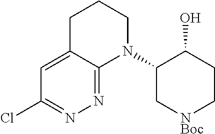 [SMILES: CC(C)(C)OC(=O)N1CC[C@@H](O)[C@@H](N2CCCc3cc(Cl)nnc32)C1]

Step 1: tert-butyl (3S,4R)-4-hydroxy-3-(pent-4-yn-1-ylamino)piperidine-1-carboxylate: A suspension of tert-butyl (3S,4R)-3-amino-4-hydroxypiperidine-1-carboxylate (Combi-Blocks, 194 mg, 0.898 mmol) and K₂CO₃ (169 mg, 1.22 mmol) in MeCN (3.3 mL) was treated with 5-bromo-1-pentyne (Enamine, 95 μL, 0.816 mmol), and the mixture was heated to 80° C. for 3 h with stirring. Then the reaction mixture was cooled to room temperature, filtered, and concentrated to give the title compound, which was used in the subsequent reaction without further purification. LCMS [M+H]⁺=283.4 (calcd. 283.2).

Step 2: tert-butyl (3S,4R)-3-(3-chloro-6,7-dihydropyrido[2,3-c]pyridazin-8(5H)-yl)-4-hydroxypiperidine-1-carboxylate: A solution of tert-butyl (3S,4R)-4-hydroxy-3-(pent-4-yn-1-ylamino)piperidine-1-carboxylate (230 mg, 0.814 mmol) in THF (2.3 mL) in a sealed tube was treated with Et₃N (125 μL. 0.896 mmol) and 3,6-dichloro-1,2,4,5-tetrazine (Pharmablock, 123 mg, 0.814 mmol). The reaction mixture was heated to 105° C. for 16 h. After cooling to room temperature, the reaction mixture was concentrated. The resulting crude residue was purified by silica gel chromatography [(25% EtOH in EtOAc):hexanes] to give the title compound. LCMS [M+H]⁺=369.2 (calcd. 369.2).

TABLE 1
The following intermediates were prepared using a procedure similar to that described
for Intermediate 1 using the appropriate starting materials.

| | | | LCMS |
|---|---|---|---|
| Intermediate | Structure | Name | [M + H]⁺ |
| 2 | 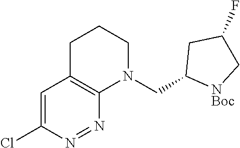 [SMILES: CC(C)(C)OC(=O)N1C[C@@H](F)C[C@H]1CN1CCCc2cc(Cl)nnc21] | tert-butyl (2S,4S)-2- ((3-chloro-6,7- dihydropyrido[2,3- c]pyridazin-8(5H)- yl)methyl)-4- fluoropyrrolidine-1- carboxylate | Calcd. 371.2, Found 371.2 |
| 3 | 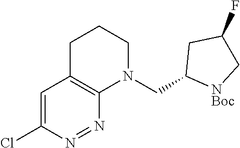 [SMILES: CC(C)(C)OC(=O)N1C[C@H](F)C[C@H]1CN1CCCc2cc(Cl)nnc21] | tert-butyl (2S,4R)-2- ((3-chloro-6,7- dihydropyrido[2,3- c]pyridazin-8(5H)- yl)methyl)-4- fluoropyrrolidine-1- carboxylate | Calcd. 371.2, Found 371.2 |

### Intermediate 4

### tert-butyl ((R)-3-chloro-8-((Is 3S)-3-hydroxy-3-methylcyclobutyl)-5,6,7,8-tetrahydropyrido[2,3-c]pyridazin-6-yl)carbamate

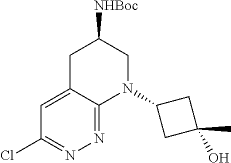 [SMILES: CC(C)(C)OC(=O)N[C@@H]1Cc2cc(Cl)nnc2N([C@H]2C[C@@](C)(O)C2)C1]

Step 1: tert-butyl ((R)-1-(((1s,3S)-3-hydroxy-3-methylcyclobutyl)amino)pent-4-yn-2-yl)carbamate: A stirring solution of tert-butyl (R)-(1-hydroxypent-4-yn-2-yl)carbamate (Ambeed, 100 mg, 0.502 mmol) and TEMPO (7.84 mg, 0.050 mmol) in DCM (3.3 mL) was treated with PIDA (Aldrich, 178 mg, 0.552 mmol), and the resulting reaction mixture was stirred at 25° C. for 1 h. Then 3-amino-1-methylcyclobutan-1-ol (Pharmablock, 51 mg, 0.502 mmol) and sodium triacetoxyborohydride (213 mg, 1.00 mmol) were added, and the reaction mixture was stirred at 25° C. for 1 h. The reaction mixture was then quenched with water and washed with DCM (×3). The aqueous layer was separated, treated with 1 M HCl (5 mL), and eluted through a BiotageR SCX-2 ion exchange column (eluent MeOH; then 7 M ammonia in MeOH). The ammonia layer was concentrated to give the title compound, which was used in the subsequent reaction without further purification. LCMS [M+H]⁺=283.4 (calcd. 283.2).

Step 2: tert-butyl ((R)-3-chloro-8-((1s,3S)-3-hydroxy-3-methylcyclobutyl)-5,6,7,8-tetrahydropyrido[2,3-c]pyridazin-6-yl)carbamate: A solution of tert-butyl ((R)-1-(((1s,3S)-3-hydroxy-3-methylcyclobutyl)amino)pent-4-yn-2-yl)carbamate (65 mg, 0.230 mmol) in THF (0.66 mL) in a sealed tube was treated with Et₃N (35.3 μL, 0.253 mmol) and 3,6-dichloro-1,2,4,5-tetrazine (Pharmablock, 34.7 mg, 0.230 mmol). The reaction mixture was heated to 105° C. for 16 h. After cooling to room temperature, the reaction mixture was concentrated. The resulting crude residue was purified by silica gel chromatography [(25% EtOH in EtOAc):hexanes] to give the title compound. LCMS [M+H]⁺=369.2 (calcd. 369.2).

### Intermediate 5

### (R)-3-chloro-8-(1-ethylpiperidin-3-yl)-4-methyl-5,6,7,8-tetrahydropyrido[2,3-c]pyridazine

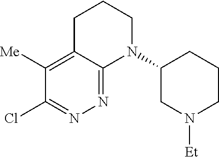 [SMILES: CCN1CCC[C@@H](N2CCCc3c2nnc(Cl)c3C)C1]

Step 1: (R)-1-ethyl-N-(hex-4-yn-1-yl)piperidin-3-amine: A suspension of (R)-1-ethylpiperidin-3-amine (Enamine, 876 mg, 6.83 mmol) and K₂CO₃ (1.29 g, 9.31 mmol) in MeCN (15.5 mL) was treated with 6-bromohex-2-yne (Enamine, 625 μl, 6.21 mmol) and the mixture was heated to 80° C. for 3 h with stirring. Then the reaction mixture was cooled to room temperature, filtered, and concentrated to give the title compound, which was used in the subsequent reaction without further purification. LCMS [M+H]⁺=209.3 (calcd. 209.2).

Step 2: (R)-6-chloro-N-(1-ethylpiperidin-3-yl)-N-(hex-4-yn-1-yl)-1,2,4,5-tetrazin-3-amine: A solution of (R)-1-ethyl-N-(hex-4-yn-1-yl)piperidin-3-amine (1.30 g, 6.23 mmol) in THF (15 mL) in a sealed vial was treated with Et₃N (813 μL, 5.83 mmol) and 3,6-dichloro-1.2.4,5-tetrazine (Pharmablock, 800 mg, 5.30 mmol). The reaction mixture was heated to 110° C. for 16 h. The reaction mixture was then cooled to room temperature, filtered, and concentrated to give the title compound, which was used in the subsequent reaction without further purification. LCMS [M+H]⁺=323.2 (calcd. 323.2).

Step 3: (R)-3-chloro-8-(1-ethylpiperidin-3-yl)-4-methyl-5,6,7,8-tetrahydropyrido[2,3-c]pyridazine: A solution of (R)-6-chloro-N-(1-ethylpiperidin-3-yl)-N-(hex-4-yn-1-yl)-1,2,4,5-tetrazin-3-amine (50 mg, 0.155 mmol) in NMP (1.5 mL) was heated to 170° C. for 12 h. Then the reaction mixture was cooled to room temperature, filtered, and purified via preparative reverse phase HPLC (C₁₈ stationary phase, MeCN/water+0.1% TFA) to give the title compound.

LCMS [M+H]⁺=295.2, (calcd. 295.2).

### Intermediate 6

### 2-(2-(ethoxymethoxy)-6-methyl-4-(trifluoromethyl)phenyl)-4,4,5,5-tetramethyl-1,3,2-dioxaborolane

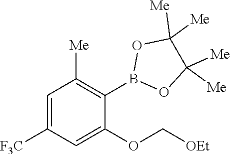 [SMILES: CCOCOc1cc(C(F)(F)F)cc(C)c1B1OC(C)(C)C(C)(C)O1]

Step 1: 3-Methyl-5-(trifluoromethyl)phenol: To a solution of 3-bromo-5-(trifluoromethyl)phenol (Carbosynth, 500 g, 2.07 mol), K₂CO₃ (859 g. 6.22 mol) and Pd(dppf)Cl₂ (75.8 g, 103.7 mmol) in 1,4-dioxane (7.5 L) under a N₂ atmosphere, was added trimethyl-1.3.5.2.4,6-trioxatriborinane (Aldrich, 1.04 kg, 4.15 mol, 50 wt % in THF) portion-wise. The resulting mixture was stirred for 12 hours at 100° C., followed by cooling to 25° C. The reaction mixture was then quenched with ice water at 0° C., diluted with EtOAc, and the layers were separated. The organic layer was washed with brine, dried over anhydrous Na₂SO₄ and concentrated. The resulting crude residue was purified by silica gel chromatography (EtOAc:petroleum ether) to give the title compound.

LCMS [M−H]⁻=175.1 (calcd. 175.0).

Step 2: 2-Iodo-3-methyl-5-(trifluoromethyl)phenol: NaH (128.5 g, 3.21 mol, 60 wt %) was added at 0° C. to a stirring solution of 3-methyl-5-(trifluoromethyl)phenol (283 g, 1.61 mol) in toluene (1.42 L) under a N₂ atmosphere. The resulting mixture was stirred at 0° C. for 30 minutes, followed by the portion-wise addition of a solution of 12 (306.1 g. 1.21 mmol) in toluene (5.66 L). The resulting mixture was stirred at 20° C. for 3 hours, and then quenched by pouring onto a water/ice bath. The mixture was diluted with EtOAc, and the layers separated. The organic layer was washed with brine, dried over anhydrous Na₂SO₄, and the solvents removed under reduced pressure. The resulting crude residue containing the title compound was carried forward without further purification.

Step 3: 1-(Ethoxymethoxy)-2-iodo-3-methyl-5-(trifluoromethyl)benzene: Chloromethyl ethyl ether (290 g, 3.07 mol) was added at 0° C. to a stirring solution of 2-iodo-3-methyl-5-(trifluoromethyl)phenol (463 g, 1.53 mol) and Cs₂CO₃ (999 g, 3.07 mmol) in DMF (4.6 L) under a N₂ atmosphere. The resulting mixture was stirred for 8 h at room temperature, then cooled to 0° C., and quenched with the addition of ice water. The mixture was diluted with EtOAc, and the layers separated. The organic layer washed with brine, dried over anhydrous Na₂SO₄, and the solvents were removed under reduced pressure. The resulting crude residue was purified by silica gel chromatography (EtOAc:petroleum ether) to give the title compound. ¹H NMR (300 MHz. DMSO-d₆) δ 7.55 (s. TH), 7.18 (s. 1H), 5.42 (s, 2H), 3.75-3.65 (m, 2H). 2.50 (s. 3H), 1.21-1.10 (m, 3H).

Step 4: 2-(2-(Ethoxymethoxy)-6-methyl-4-(trifluoromethyl)phenyl)-4,4,5,5-tetramethyl-1,3,2-dioxaborolane: A mixture of 1-(ethoxymethoxy)-2-iodo-3-methyl-5-(trifluoromethyl)benzene (330 g. 916.4 mmol), B₂pin₂ (469 g. 3.67 mol), Et₃N (556 g, 5.50 mol). Pd(OAc)₂ (10.3 g, 45.8 mmol), biphenyl-2-yl-diclohexylphosphine (32.1 g, 91.6 mmol) in 1,4-dioxane (3.3 L) was placed under a N₂ atomsphere. The reaction mixture was stirred for 6 h at 100° C., then cooled to 25° C., and quenched with ice water. The resulting mixture was filtered, and the solid residue washed with EtOAc. The layers in the filtrate were separated. The organic layer washed with brine, dried over anhydrous Na₂SO₄ and concentrated. The resulting crude residue was purified by silica gel chromatography (EtOAc:petroleum ether), and the desired fractions were concentrated. The resulting solid was dissolved in hexanes and stirred for 5 minutes at −30° C. The resulting precipitated solids were collected by filtration to afford the title compound. ¹H NMR (300 MHz, CDCl₃) δ 7.13-7.03 (m, 2H), 5.23 (s, 2H), 3.74 (q, J=7.1 Hz, 2H), 2.41 (s, 3H), 1.41 (s, 12H), 1.24 (t. J=7.1 Hz. 3H).

### Intermediate 7

### 2-(2-(difluoromethoxy)-4-(trifluoromethyl)phenyl)-4,4,5,5-tetramethyl-1,3,2-dioxaborolane

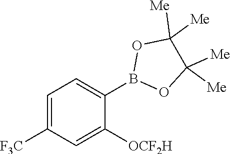

A solution of 1-bromo-2-(difluoromethoxy)-4-(trifluoromethyl)benzene (Enamine, 200 mg, 0.687 mmol) in toluene (5 mL) was treated with B₂pin₂ (0.262 g, 1.031 mmol), KOAc (0.202 g, 2.062 mmol) and PdCl₂(dppf) (0.050 g, 0.069 mmol). The resulting mixture was degassed, and then stirred at 85° C. under N₂ for 13 h. After cooling to room temperature, the reaction mixture was concentrated, and the resulting crude residue was purified by silica gel chromatography (EtOAc:petroleum ether) to give the title compound. ¹H NMR (400 MHz, CDCl₃) δ 7.88 (d, J=7.6 Hz, 1H), 7.51 (d, J=7.7 Hz, 1H), 7.41 (s, 1H), 6.56 (t, J=58.0 Hz, 1H), 1.37 (s, 12H).

### Intermediate 8

### 2-(difluoromethoxy)-3-(4,4,5,5-tetramethyl-1,3,2-dioxaborolan-2-yl)-6-(trifluoromethyl)pyridine

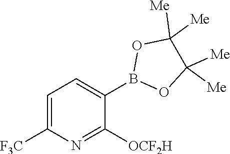

Step 1: 3-iodo-6-(trifluoromethyl)pyridin-2(H)-one: A solution of K₂CO₃ (25.3 g, 183 mmol) in water (100 mL) was treated with 6-(trifluoromethyl)pyridin-2(1H)-one (Ambeed, 10.0 g, 61.4 mmol) and I₂ (23.3 g, 91.7 mmol). The resulting reaction mixture was stirred at 25° C. for 24 hours. The reaction mixture was then slowly quenched with concentrated HCl until vigorous bubbling stopped. The mixture was then acidified to pH 3 with 1 M HCl. Then the mixture was extracted with DCM (×3), dried over Na₂SO₄, filtered, and concentrated to give the title compound, which was used in the subsequent reaction without further purification. LCMS [M+H]⁺=289.9 (calcd. 289.9).

Step 2: 2-(difluoromethoxy)-3-iodo-6-(trifluoromethyl)pyridine: A stirring solution of 3-iodo-6-(trifluoromethyl)pyridin-2(1H)-one (2.00 g, 6.92 mmol) and (bromodifluoromethyl)trimethyl-silane (Pharmablock, 2.15 mL, 13.8 mmol) in MeCN (40 mL) was cooled to 0° C. Then a solution of KOH (2.33 g, 41.5 mmol) in water (10 mL) was added dropwise. The resulting reaction mixture was stirred for 30 min at 0° C. Then the reaction mixture was diluted with EtOAc, and the layers separated. The aqueous layer extracted with EtOAc (×2). The combined organic layers were dried over MgSO₄, filtered, and concentrated. The resulting crude residue was purified by silica gel chromatography (EtOAc:hexanes) to give the title compound. ¹H NMR (500 MHz, CDCl₃) δ 8.34 (d, J=7.8 Hz, 1H). 7.48 (t. J=71.5 Hz, 1H), 7.23 (d, J=7.8 Hz, 1H).

Step 3: 2-(difluoromethoxy)-3-(4,4,5,5-tetramethyl-1.3,2-dioxaborolan-2-yl)-6-(trifluoromethyl)pyridine: A stirring mixture of B₂pin₂ (1.85 g, 7.29 mmol), PCy₃ Pd G3 (Aldrich, 99 mg, 0.152 mmol), and KOAc (1.79 g, 18.2 mmol) in cyclopentyl methyl ether (31 mL) was degassed with N₂ for 10 min. Then 2-(difluoromethoxy)-3-iodo-6-(trifluoromethyl)pyridine (2.06 g, 6.08 mmol) was added, and the mixture was degassed by bubbling with N₂ for an additional 5 minutes. The reaction was then stirred at 85° C. for 16 h. Then the reaction mixture was cooled to room temperature, additional PCy₃ Pd G3 (Aldrich, 99 mg, 0.152 mmol) was added, and the reaction mixture was degassed with N₂ for 10 minutes. Then the reaction was stirred at 85° C. for 24 h. After cooling to room temperature, the reaction mixture was diluted with EtOAc, filtered over Celite™, and concentrated to give the title compound, which was used in the subsequent step without further purification. ¹H NMR (500 MHz, CDCl₃) δ 8.24 (d, J=7.4 Hz, 1H), 7.52 (d, J=72.4 Hz, 1H), 7.44 (d, J=7.4 Hz, 1H), 1.37 (s, 12H).

### Intermediate 9

### (3S,4R)-3-(3-(2-hydroxy-6-methyl-4-(trifluoromethyl)phenyl)-6,7-dihydropyrido[2,3-c]pyridazin-8(5H)-yl)piperidin-4-ol

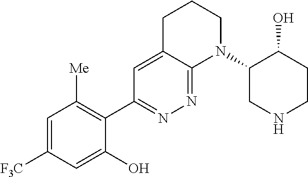 [SMILES: Cc1cc(C(F)(F)F)cc(O)c1-c1cc2c(nn1)N([C@H]1CNCC[C@H]1O)CCC2]

Step 1: tert-butyl 3-(3-(2-(ethoxymethoxy)-6-methyl-4-(trifluoromethyl)phenyl)-6,7-dihydropyrido[2,3-c]pyridazin-8(5H)-yl)-4-hydroxypiperidine-1-carboxylate: A vial was charged with tert-butyl 3-(3-chloro-6,7-dihydropyrido[2,3-c]pyridazin-8(5H)-yl)-4-hv droxypiperidine-1-carboxylate (Intermediate 1, 35 mg, 0.095 mmol), 2-(2-(ethoxymethoxy)-6-methyl-4-(trifluoromethyl)phenyl)-4,4,5,5-tetramethyl-1,3,2-dioxaborolane (Intermediate 6, 51 mg, 0.142 mmol), XPhos Pd G3 (6.4 mg, 0.0076 mmol), and potassium carbonate (66 mg, 0.474 mmol), and then evacuated and backfilled with N₂ (×3). In a separate vial, a solvent mixture of 1,4-dioxane (0.5 mL) and water (0.13 mL) was sparged with N₂ for 15 minutes and then added to the reaction vial. The reaction mixture was heated to 100° C. for 3 hours, then cooled to room temperature, and diluted with water and DCM. The layers were separated, and the aqueous layer was extracted with DCM (×3). The combined organic layers were dried over anhydrous MgSO₄, filtered and concentrated. The resulting crude residue was purified by silica gel chromatography [(25% EtOH in EtOAc):hexanes] to give the title compound. LCMS [M+H]⁺=567.3, (calcd. 567.3).

Step 2: (3S,4R)-3-(3-(2-hydroxy-6-methyl-4-(trifluoromethyl)phenyl)-6,7-dihydropyrido[2,3-c]pyridazin-8(5H)-yl)piperidin-4-ol: A solution of tert-butyl (3S,4R)-3-(3-(2-(ethoxymethoxy)-6-methyl-4-(trifluoromethyl)phenyl)-6,7-dihydropyrido[2,3-c]pyridazin-8(5H)-yl)-4-hydroxy-piperidine-1-carboxylate (16 mg, 0.028 mmol) in 1,4-dioxane (3.8 mL) was treated with HCl (4 M in 1,4-dioxane, 35 μL, 0.141 mmol). The reaction mixture was heated to 70° C. and stirred for 3 h. After cooling to room temperature, the reaction mixture was treated with 1 M HCl and MeOH, and then eluted through a Biotage SCX-2 ion exchange column (eluent MeOH; then 7 M ammonia in MeOH). The ammonia layer was concentrated to give the title compound. LCMS [M+H]⁺=409.3, (calcd. 409.2).

TABLE 2
The following intermediate was prepared using a procedure similar to that described for
Intermediate 9 using the appropriate starting material.

| | | | LCMS |
|---|---|---|---|
| Intermediate | Structure | Name | [M + H]⁺ |
| 10 | 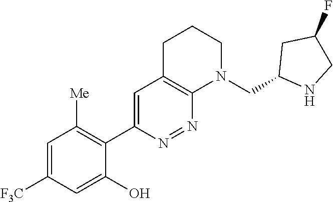 [SMILES: Cc1cc(C(F)(F)F)cc(O)c1-c1cc2c(nn1)N(C[C@@H]1C[C@@H](F)CN1)CCC2] | 2-(8-(((2S,4R)-4- fluoropyrrolidin-2- yl)methyl)-5,6,7,8- tetrahydropyrido[2, 3-c]pyridazin-3-yl)- 3-methyl-5- (trifluoro- methyl)phenol | Calcd. 411.2, Found 411.2 |

### Example 1

### 2-(8-((1s,3s)-3-hydroxy-3-methylcyclobutyl)-5,6,7,8-tetrahydropyrido[2,3-c]pyridazin-3-yl)-3-methyl-5-(trifluoromethyl)phenol

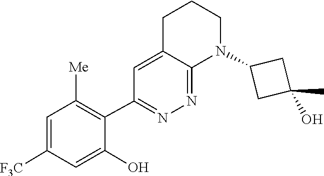 [SMILES: Cc1cc(C(F)(F)F)cc(O)c1-c1cc2c(nn1)N([C@H]1C[C@@](C)(O)C1)CCC2]

Step 1: (1s,3s)-1-methyl-3-(pent-4-yn-1-ylamino)cyclobutan-1-ol: A suspension of (1s,3s)-3-amino-1-methylcyclobutan-1-ol. HCl salt (Pharmablock, 134 mg. 0.973 mmol) and K₂CO₃ (183 mg, 1.33 mmol) in MeCN (3.5 mL) was treated with 5-bromo-1-pentyne (Enamine, 103 μL, 0.884 mmol). The mixture was heated to 80° C. for 3 hours with stirring. Then the reaction mixture was cooled to room temperature, filtered, and concentrated to give the title compound, which was used in the subsequent reaction without further purification. LCMS [M+H]~=168.2 (calcd. 168.1).

Step 2: (1s,3s)-3-(3-chloro-6,7-dihydropyrido[2,3-c]pyridazin-8(5H)-yl)-1-methylcyclobutan-1-ol: A solution of (1s,3s)-1-methyl-3-(pent-4-yn-1-ylamino)cyclobutan-1-ol (148 mg, 0.885 mmol) in THF (2.5 mL) in a sealed tube was treated with Et₃N (136 μL, 0.973 mmol) and 3,6-dichloro-1.2,4,5-tetrazine (Pharmablock, 134 mg, 0.885 mmol). The reaction mixture was heated to 105° C. for 16 h. After cooling to room temperature, the reaction mixture was concentrated. The resulting crude residue was purified by silica gel chromatography (MeOH:DCM) to give the title compound. LCMS [M+H]⁺=254.2 (calcd. 254.1).

Step 3: (1s,3s)-3-(3-(2-(ethoxymethoxy)-6-methyl-4-(trifluoromethyl)phenyl)-6,7-dihydropyrido[2,3-c]pyridazin-8(5H)-yl)-1-methylcyclobutan-1-ol: A vial was charged with (1s,3s)-3-(3-chloro-6,7-dihydropyrido[2,3-c]pyridazin-8(5H)-yl)-1-methylcyclobutan-1-ol (107 mg, 0.422 mmol), 2-(2-(ethoxymethoxy)-6-methyl-4-(trifluoromethyl)phenyl)-4,4,5,5-tetramethyl-1,3,2-dioxaborolane (Intermediate 6, 228 mg, 0.633 mmol). XPhos Pd G3 (29 mg, 0.034 mmol), and K₂CO₃ (291 mg, 2.11 mmol), then evacuated and backfilled with N₂ (×3). In a separate vial, a solvent mixture of 1,4-dioxane (2.2 mL) and water (0.6 mL) was sparged with N₂ for 15 minutes and then added to the reaction vial. The reaction mixture was heated to 100° C. for 3 hours. Then the reaction mixture was cooled to room temperature, and diluted with water and DCM. The layers were separated, and the aqueous layer was extracted with DCM (×3). The combined organic layers were dried over anhydrous MgSO₄, filtered and concentrated. The resulting crude residue was purified by silica gel chromatography (MeOH:DCM) to give the title compound.

LCMS [M+H]⁺=452.3, (calcd. 452.2).

Step 4: 2-(8-((1s,3s)-3-hydroxy-3-methylcyclobutyl)-5.6.7,8-tetrahydropyrido[2,3-c]pyridazin-3-yl)-3-methyl-5-(trifluoromethyl)phenol: A solution of (1s,3s)-3-(3-(2-(ethoxymethoxy)-6-methyl-4-(trifluoromethyl)phenyl)-6,7-dihydropyrido[2,3-c]pyridazin-8(5H)-yl)-1-methylcyclobutan-1-ol (173 mg, 0.383 mmol) in 1,4-dioxane (3.8 mL) was treated with HCl (4 M in 1,4-dioxane, 479 μL. 1.92 mmol). The reaction mixture was heated to 70° C. and stirred for 3 h. Then the reaction mixture was cooled to room temperature, and concentrated. The resulting crude residue was dissolved in DMSO, filtered, and purified via preparative reverse phase HPLC (C₁₈ stationary phase, MeCN/water+0.05% FA) to give the title compound. LCMS [M+H]⁺=394.2, (calcd. 394.2). ¹H NMR (500 MHz, DMSO-d₆) δ 7.15 (br s, 1H), 7.11 (s, 1H). 7.08 (s, 1H), 5.08 (br s. 1H). 4.86-4.76 (m. 1H), 3.53-3.47 (m. 2H), 2.75 (app t, J=6.0 Hz. 1H), 2.30-2.25 (m, 4H), 2.14 (s, 3H), 1.92-1.84 (m, 2H), 1.32 (s, 3H).

TABLE 3
The following compounds were prepared using procedures similar to those described for
Example 1 using the appropriate starting materials.

| | | | LCMS |
|---|---|---|---|
| Example | Structure | Name | [M + H]⁺ |
| 2 | 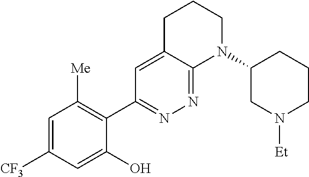 [SMILES: CCN1CCC[C@@H](N2CCCc3cc(-c4c(C)cc(C(F)(F)F)cc4O)nnc32)C1] | (R)-2-(8-(1- ethylpiperidin-3-yl)- 5,6,7,8- tetrahydropyrido[2,3- c]pyridazin-3-yl)-3- methyl-5- (trifluoromethyl)phenol | Calcd. 421.2, Found 421.4 |
| 3 | 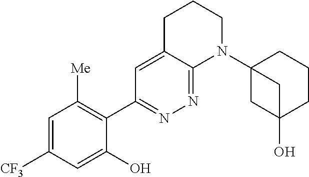 [SMILES: Cc1cc(C(F)(F)F)cc(O)c1-c1cc2c(nn1)N(C13CCCC(O)(C1)C3)CCC2] | 5-(3-(2-hydroxy-6- methyl-4- (trifluoromethyl)phenyl)- 6,7-dihydropyrido[2,3- c]pyridazin-8(5H)- yl)bicyclo[3.1.1]heptan- 1-ol | Calcd. 420.2, Found 420.2 |
| 4 | 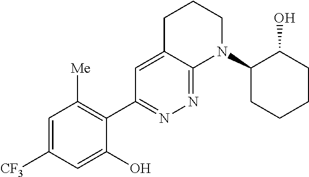 [SMILES: Cc1cc(C(F)(F)F)cc(O)c1-c1cc2c(nn1)N([C@@H]1CCCC[C@H]1O)CCC2] | 2-(8-((1R,2R)-2- hydroxycyclohexyl)- 5,6,7,8- tetrahydropyrido[2,3- c]pyridazin-3-yl)-3- methyl-5- (trifluoromethyl)phenol | Calcd. 408.2, Found 408.4 |

TABLE 4
The following compounds were prepared using procedures similar to those described for
Example 1 using the appropriate starting materials. Enantiomeric products were separated after
step 4 using chiral SFC methods specified in the table. For pairs of enantiomers, the faster-
eluting isomer is listed first.

| | | | LCMS | Chiral |
|---|---|---|---|---|
| Example | Structure | Name | [M + H]⁺ | Method |
| 5 | 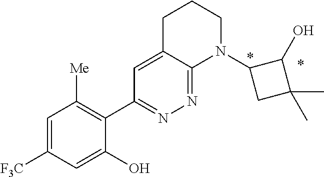 [SMILES: Cc1cc(C(F)(F)F)cc(O)c1-c1cc2c(nn1)N(C1CC(C)(C)C1O)CCC2] | 2-(8-((1S,2R or 1R,2S)-2- hydroxy-3,3- dimethyl- cyclobutyl)- 5,6,7,8- tetrahydropyrido [2,3-c]pyridazin-3- yl)-3-methyl-5- (trifluoromethyl) phenol | Calcd. 408.2, Found 408.3 | A |
| 6 | 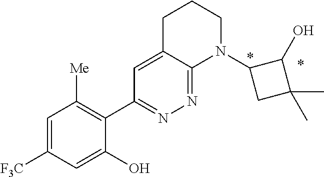 [SMILES: Cc1cc(C(F)(F)F)cc(O)c1-c1cc2c(nn1)N(C1CC(C)(C)C1O)CCC2] | 2-(8-((1R,2S or 1S,2R)-2- hydroxy-3,3- dimethyl- cyclobutyl)- 5,6,7,8- tetrahydropyrido [2,3-c]pyridazin-3- yl)-3-methyl-5- (trifluoromethyl) phenol | Calcd. 408.2, Found 408.3 | A |

### Example 7

### 2-(8-(((2S,4S)-4-fluoropyrrolidin-2-yl)methyl)-5,6,7,8-tetrahydropyrido[2,3-c]pyridazin-3-yl)-3-methyl-5-(trifluoromethyl)phenol

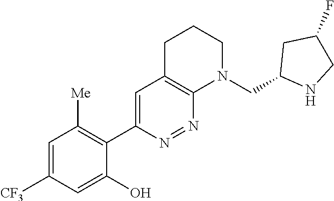 [SMILES: Cc1cc(C(F)(F)F)cc(O)c1-c1cc2c(nn1)N(C[C@@H]1C[C@H](F)CN1)CCC2]

Step 1: tert-butyl (2S,4S)-2-((3-(2-(ethoxymethoxy)-6-methyl-4-(trifluoromethyl)phenyl)-6,7-dihydropyrido[2,3-c]pyridazin-8(5H)-yl)methyl)-4-fluoropyrrolidine-1-carboxylate: A vial was charged with tert-butyl (2S,4S)-2-((3-chloro-6,7-dihydropyrido[2,3-c]pyridazin-8(5H)-yl)methyl)-4-fluoropyrrolidine-1-carboxylate (Intermediate 2, 142 mg, 0.383 mmol), 2-(2-(ethoxymethoxy)-6-methyl-4-(trifluoromethyl)phenyl)-4,4,5,5-tetramethyl-1.3,2-dioxaborolane (Intermediate 6, 207 mg. 0.574 mmol), XPhos Pd G3 (26 mg. 0.031 mmol), and K₂CO₃ (265 mg, 1.92 mmol), then evacuated and backfilled with N₂ (×3). In a separate vial, a solvent mixture of 1,4-dioxane (2.0 mL) and water (0.5 mL) was sparged with N₂ for 15 minutes, and then added to the reaction vial. The reaction mixture was heated to 100° C. for 6 h. Then the reaction mixture was cooled to room temperature, and diluted with water and DCM. The layers were separated, and the aqueous layer was extracted with DCM (×3). The combined organic layers were dried over anhydrous MgSO₄, filtered, and concentrated. The resulting crude residue was purified by silica gel chromatography [(25% EtOH in EtOAc):hexanes] to give the title compound. LCMS [M+H]⁺=569.4, (calcd. 569.3).

Step 2: 2-(8-(((2S,4S)-4-fluoropyrrolidin-2-yl)methyl)-5,6,7,8-tetrahydropyrido[2,3-c]pyridazin-3-yl)-3-methyl-5-(trifluoromethyl)phenol: A solution of tert-butyl (2S,4S)-2-((3-(2-(ethoxymethoxy)-6-methyl-4-(trifluoromethyl)phenyl)-6,7-dihydropyrido[2,3-c]pyridazin-8(5H)-yl)methyl)-4-fluoropyrrolidine-1-carboxylate (200 mg, 0.352 mmol) in 1,4-dioxane (3.5 mL) was treated with HCl (4 M in 1,4-dioxane, 0.44 mL, 1.76 mmol). The reaction mixture was heated to 70° C. and stirred for 3 h. Then the reaction mixture was cooled to room temperature, and concentrated. The crude residue was dissolved in DMSO, filtered, and purified via preparative reverse phase HPLC (C₁₈ stationary phase, MeCN/water+0.05% FA) to give the title compound. LCMS [M+H]⁺=411.2, (calcd. 411.2). ¹H NMR (500 MHz, DMSO-d₆) δ 7.09 (s, 1H), 7.05 (s, 1H), 7.03 (s, 1H), 5.24 (d, J=55.9 Hz, 1H), 3.84 (dd, J=13.6, 5.0 Hz, 1H), 3.63 (dd, J=13.2, 8.6 Hz, 2H). 3.58-3.49 (m. 2H), 3.12 (dd, J=22.4, 12.7 Hz, 1H). 2.90 (ddd, J=34.1, 12.8, 4.3 Hz, 1H), 2.75 (app t, J=6.1 Hz, 2H), 2.28-2.11 (m, 1H), 2.14 (s, 3H) 1.93-1.77 (m, 2H), 1.68 (ddd, J=31.4, 14.5, 6.2 Hz, 1H).

TABLE 5
The following compound was prepared using a procedure similar to that described for
Example 7 using the appropriate starting material.

| | | | LCMS |
|---|---|---|---|
| Example | Structure | Name | [M + H]⁺ |
| 8 | 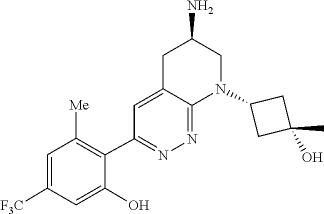 [SMILES: Cc1cc(C(F)(F)F)cc(O)c1-c1cc2c(nn1)N([C@H]1C[C@@](C)(O)C1)C[C@H](N)C2] | 2-((R)-6-amino-8- ((1s,3S)-3-hydroxy-3- methylcyclobutyl)- 5,6,7,8- tetrahydropyrido[2,3- c]pyridazin-3-yl)-3- methyl-5- (trifluoromethyl)phenol | Calcd. 409.2, Found 409.3 |

### Example 9

### (R)-2-(8-(1-ethylpiperidin-3-yl)-4-methyl-5,6,7,8-tetrahydropyrido[2,3-c]pyridazin-3-yl)-5-(trifluoromethyl)phenol

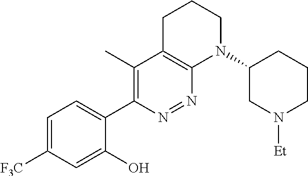 [SMILES: CCN1CCC[C@@H](N2CCCc3c2nnc(-c2ccc(C(F)(F)F)cc2O)c3C)C1]

A vial was charged with (R)-3-chloro-8-(1-ethylpiperidin-3-yl)-4-methyl-5,6,7,8-tetrahydropyrido[2,3-c]pyridazine (Intermediate 5, 33 mg, 0.112 mmol), (2-hydroxy-4-(trifluoromethyl)phenyl)boronic acid (Combi-Blocks, 43.8 mg, 0.213 mmol), XPhos Pd G3 (7.58 mg, 8.95 μmol), and K₂CO₃ (77 mg, 0.560 mmol), then evacuated and backfilled with N₂ (×3). In a separate vial, a solvent mixture of DMSO (900 μL) and water (225 μL) was sparged with N₂ for 15 min and then added to the reaction vial. The reaction mixture was heated to 100° C. for 2 h. Then the reaction mixture was cooled to room temperature, filtered, and purified via preparative reverse phase HPLC (C₁₈ stationary phase, MeCN/water+0.05% FA) to afford the title compound. LCMS [M+H]⁺=421.4, (calcd. 421.2). ¹H NMR (500 MHz, DMSO-d₆) δ 7.36 (d, J=8.3 Hz. 1H), 7.23-7.19 (m, 2H), 5.04-4.95 (m, 1H), 2.88-2.80 (m, 2H), 2.69-2.60 (m, 3H). 2.40-2.29 (m, 3H), 2.00 (app t, J=10.6 Hz, 1H), 1.90 (s, 3H), 1.89-1.52 (m, 7H), 0.99 (t, J=7.2 Hz, 3H).

TABLE 6
The following compounds were prepared using a procedure similar to that described for
Example 9 using the appropriate starting materials.

| | | | LCMS |
|---|---|---|---|
| Example | Structure | Name | [M + H]⁺ |
| 10 | 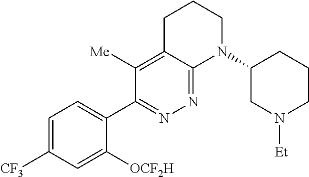 | (R)-3-(2- (difluoromethoxy)-4- (trifluoromethyl)phenyl)- 8-(1-ethylpiperidin-3-yl)- 4-methyl-5,6,7,8- tetrahydropyrido[2,3- c]pyridazine | Calcd. 471.2, Found 471.3 |
| 11 | 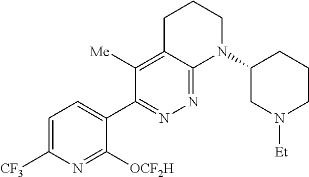 | (R)-3-(2- (difluoromethoxy)-6- (trifluoromethyl)pyridin- 3-yl)-8-(1-ethylpiperidin- 3-yl)-4-methyl-5,6,7,8- tetrahydropyrido[2,3- c]pyridazine | Calcd. 472.2, Found 472.3 |
| 12 | 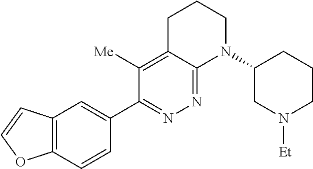 [SMILES: CCN1CCC[C@@H](N2CCCc3c2nnc(-c2ccc4occc4c2)c3C)C1] | (R)-3-(benzofuran-5-yl)- 8-(1-ethylpiperidin-3-yl)- 4-methyl-5,6,7,8- tetrahydropyrido[2,3- c]pyridazine | Calcd. 377.2, Found 377.3 |

### Example 13

### 2-(8-(((2S,4S)-4-fluoro-1-methylpyrrolidin-2-yl)methyl)-5,6,7,8-tetrahydropyrido[2,3-c]pyridazin-3-yl)-3-methyl-5-(trifluoromethyl)phenol

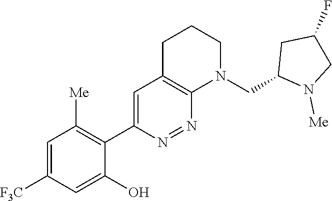 [SMILES: Cc1cc(C(F)(F)F)cc(O)c1-c1cc2c(nn1)N(C[C@@H]1C[C@H](F)CN1C)CCC2]

A solution of 2-(8-(((2S,4S)-4-fluoropyrrolidin-2-yl)methyl)-5,6,7,8-tetrahydropyrido[2,3-c]pyridazin-3-yl)-3-methyl-5-(trifluoromethyl)phenol (Example 7, 32 mg, 0.078 mmol) in MeOH (390 μL) and THF (390 μL) was treated with formaldehyde (37% in water, 35 μL, 0.468 mmol) and sodium triacetoxyborohydride (50 mg, 0.234 mmol). The resulting mixture was stirred for 2 h at 25° C. Then the reaction was quenched with AcOH (15 μL), and concentrated. The resulting residue was taken up in DMSO, filtered and purified via preparative reverse phase HPLC (C₁₈ stationary phase. MeCN/water+0.05% FA) to give the title compound. LCMS [M+H]⁺=425.3, (calcd. 425.2). ¹H NMR (500 MHz, DMSO-d₆) δ 7.09 (s, 1H), 7.07 (s, 1H), 7.03 (s, 1H), 5.11 (d, J 55.4 Hz, 1H), 4.10 (dd, J=13.8, 4.2 Hz, 1H), 3.65-3.59 (m, 1H), 3.54 (ddd, J=18.0, 13.2, 6.3 Hz, 2H), 3.17 (dd, J=19.3, 11.6 Hz, 1H), 2.75 (t, J=5.9 Hz, 2H), 2.65-2.57 (m, 1H), 2.46-2.35 (m, 1H), 2.34 (s, 3H). 2.33-2.28 (m, 1H). 2.24 (dd, J=11.6, 4.1 Hz, 1H), 2.14 (s, 3H), 1.92-1.77 (m, 3H).

TABLE 7
The following compounds were prepared using a procedure similar to that described for
Example 13 using the appropriate starting materials.

| | | | LCMS |
|---|---|---|---|
| Example | Structure | Name | [M + H]⁺ |
| 14 | 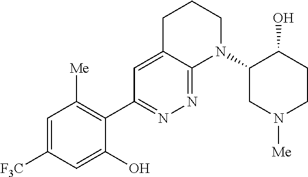 [SMILES: Cc1cc(C(F)(F)F)cc(O)c1-c1cc2c(nn1)N([C@H]1CN(C)CC[C@H]1O)CCC2] | (3S,4R)-3-(3-(2-hydroxy- 6-methyl-4- (trifluoromethyl)phenyl)- 6,7-dihydropyrido[2,3- c]pyridazin-8(5H)-yl)-1- methylpiperidin-4-ol | Calcd. 423.2, Found 423.3 |
| 15 | 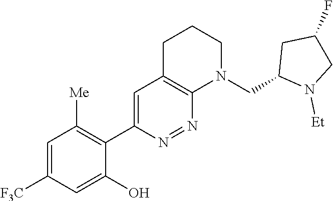 [SMILES: CCN1C[C@@H](F)C[C@H]1CN1CCCc2cc(-c3c(C)cc(C(F)(F)F)cc3O)nnc21] | 2-(8-(((2S,4S)-1-ethyl-4- fluoropyrrolidin-2- yl)methyl)-5,6,7,8- tetrahydropyrido[2,3- c]pyridazin-3-yl)-3- methyl-5- (trifluoromethyl)phenol | Calcd. 439.2, Found 439.2 |

### Example 16

### 1-((2S,4R)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)ethan-1-one

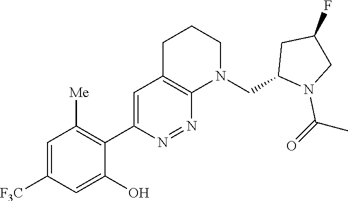 [SMILES: CC(=O)N1C[C@H](F)C[C@H]1CN1CCCc2cc(-c3c(C)cc(C(F)(F)F)cc3O)nnc21]

A solution of 2-(8-(((2S,4R)-4-fluoropyrrolidin-2-yl)methyl)-5,6,7,8-tetrahydropyrido[2,3-c]pyridazin-3-yl)-3-methyl-5-(trifluoromethyl)phenol (Intermediate 10, 18 mg. 0.044 mmol) in DMF (440 μL) was treated with AcOH (2.5 μL, 0.044 mmol), DIPEA (19 μL, 0.110 mmol), and HATU (25 mg, 0.066 mmol). The resulting mixture was stirred for 45 min at 25° C. Then the reaction mixture was filtered, washed with DMSO, and the resulting filtrate purified via preparative reverse phase HPLC (C₁₈ stationary phase, MeCN/water+0.05% FA) to give the title compound. LCMS [M+H]⁺=453.4, (calcd. 453.2). ¹H NMR (500 MHz, DMSO-d₆) δ 8.46 (s, 1H), 7.07 (s, 1H), 7.04 (s, 1H), 7.02 (s, 1H), 5.44-5.23 (m, 1H), 4.65-4.48 (m, 1H), 4.13-3.98 (m, 1H), 3.84-3.63 (m, 21H). 3.56-3.39 (m, 1H). 2.78-2.69 (m, 2H). 2.41-2.15 (in. 2H), 2.13-2.09 (m, 3H), 2.05-1.90 (m, 3H), 1.97-1.79 (m, 2H).

Note: two protons are overlapping with the H₂O peak in the ¹1H NMR spectra (3.35 ppm).

TABLE 8
The following compounds were prepared using a procedure similar to that described for
Example 16 using the appropriate starting materials.

| | | | LCMS |
|---|---|---|---|
| Example | Structure | Name | [M + H]⁺ |
| 17 | 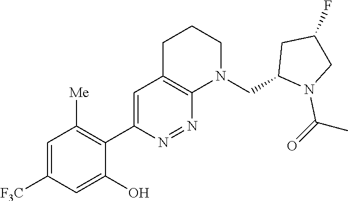 [SMILES: CC(=O)N1C[C@@H](F)C[C@H]1CN1CCCc2cc(-c3c(C)cc(C(F)(F)F)cc3O)nnc21] | 1-((2S,4S)-4-fluoro-2- ((3-(2-hydroxy-6- methyl-4- (trifluoromethyl)phenyl)- 6,7-dihydropyrido[2,3- c]pyridazin-8(5H)- yl)methyl)pyrrolidin-1- yl)ethan-1-one | Calcd. 453.3, Found 453.2 |
| 18 | 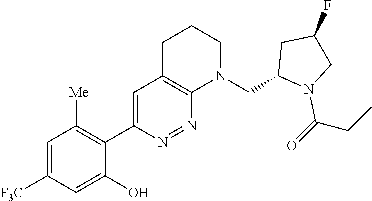 [SMILES: CCC(=O)N1C[C@H](F)C[C@H]1CN1CCCc2cc(-c3c(C)cc(C(F)(F)F)cc3O)nnc21] | 1-((2S,4R)-4-fluoro-2- ((3-(2-hydroxy-6- methyl-4- (trifluoromethyl)phenyl)- 6,7-dihydropyrido[2,3- c]pyridazin-8(5H)- yl)methyl)pyrrolidin-1- yl)propan-1-one | Calcd. 467.2, Found 467.4 |
| 19 | 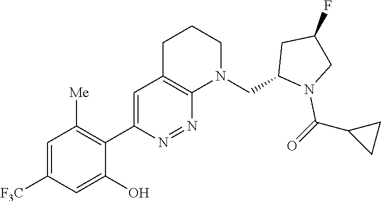 [SMILES: Cc1cc(C(F)(F)F)cc(O)c1-c1cc2c(nn1)N(C[C@@H]1C[C@@H](F)CN1C(=O)C1CC1)CCC2] | cyclopropyl((2S,4R)-4- fluoro-2-((3-(2- hydroxy-6-methyl-4- (trifluoromethyl)phenyl)- 6,7-dihydropyrido[2,3- c]pyridazin-8(5H)- yl)methyl)pyrrolidin-1- yl)methanone | Calcd. 479.2, Found 479.2 |
| 20 | 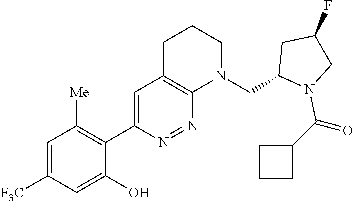 [SMILES: Cc1cc(C(F)(F)F)cc(O)c1-c1cc2c(nn1)N(C[C@@H]1C[C@@H](F)CN1C(=O)C1CCC1)CCC2] | cyclobutyl((2S,4R)-4- fluoro-2-((3-(2- hydroxy-6-methyl-4- (trifluoromethyl)phenyl)- 6,7-dihydropyrido[2,3- c]pyridazin-8(5H)- yl)methyl)pyrrolidin-1- yl)methanone | Calcd. 493.2, Found 493.4 |

### Example 21

### 2-(8-(((2S,4R)-4-fluoro-1-(methylsulfonyl)pyrrolidin-2-yl)methyl)-5,6,7,8-tetrahydropyrido[2,3-c]pyridazin-3-yl)-3-methyl-5-(trifluoromethyl)phenol

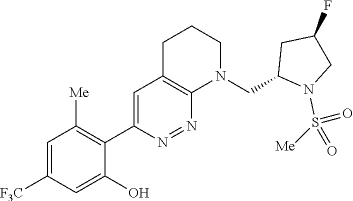 [SMILES: Cc1cc(C(F)(F)F)cc(O)c1-c1cc2c(nn1)N(C[C@@H]1C[C@@H](F)CN1S(C)(=O)=O)CCC2]

A solution of 2-(8-(((2S,4R)-4-fluoropyrrolidin-2-yl)methyl)-5,6,7,8-tetrahydropyrido[2,3-c]pyridazin-3-yl)-3-methyl-5-(trifluoromethyl)phenol (Intermediate 10, 18 mg, 0.044 mmol) in DCM (440 μL) was treated with Et₃N (31 μL, 0.219 mmol) and MsCl (14 μL, 0.175 mmol). The resulting mixture was stirred for 1.5 h at 25° C. Then hydrazine hydrate (0.6 mL) was added, and the reaction mixture was heated to 60° C. for 16 h. After cooling to room temperature, the reaction mixture was concentrated. The resulting crude residue was dissolved in DMSO, filtered, and purified via preparative reverse phase HPLC (C₁₈ stationary phase, MeCN/water+0.05% FA) to give the title compound. LCMS [M+H]⁺=489.4, (calcd. 489.2). ¹H NMR (500 MHz. DMSO-d₆) δ 7.08 (s. 1H), 7.04-7.01 (m. 2H), 5.26 (d. J=52.8 Hz, 1H). 4.33-4.26 (m, 1H). 4.01 (dd, J=13.9, 5.1 Hz, 1H), 3.87 (dd,J=14.0, 6.1 Hz, 1H), 3.73 (dd, J=19.9, 15.3 Hz, 1H), 2.89 (s, 3H), 2.73 (app t, J=6.1 Hz, 2H), 2.40-2.28 (m, 1H), 2.17-2.08 (m, 1H), 2.11 (s, 3H), 2.08-2.00 (m. 1H), 1.90-1.82 (m, 2H).

Note: two protons are overlapping with the H₂O peak in the ¹H NMR spectra (3.35 ppm).

TABLE 9
The following compound was prepared using a procedure similar to that described for
Example 21 using the appropriate starting material.

| | | | LCMS |
|---|---|---|---|
| Example | Structure | Name | [M + H]⁺ |
| 22 | 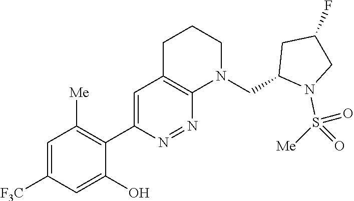 [SMILES: Cc1cc(C(F)(F)F)cc(O)c1-c1cc2c(nn1)N(C[C@@H]1C[C@H](F)CN1S(C)(=O)=O)CCC2] | 2-(8-(((2S,4S)-4-fluoro-1- (methylsulfonyl)pyrrolidin- 2-yl)methyl)-5,6,7,8- tetrahydropyrido[2,3- c]pyridazin-3-yl)-3- methyl-5- (trifluoromethyl)phenol | Calcd. 489.2, Found 489.3 |

### Example of a Pharmaceutical Composition

As a specific embodiment of an oral pharmaceutical composition, a 100 mg potency tablet is composed of 100 mg of any one of the Examples, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

### Biological Assay

Activation of the canonical NLRP3 inflammasome requires two steps, priming and activation. A priming signal such as a pathogen activated molecular patterns (PAMPs) or danger-activated molecular patterns (DAMPs) are recognized by Toll-like receptors leads to nuclear factor kappa B (NF-KB)-mediated signaling. This in turn, up-regulates transcription of inflammasome-related components, including inactive NLRP3 and prolL-1β (Bauernfeind et al., J. Immunol. 2009, 183, 787-791: Franchi et al., Nat. Immunol. 2012, 13, 325-332: Franchi et al., J. Immunol. 2014, 193, 4214-4222). The second step is activation which induces oligomerization ofNLRP3 and subsequent assembly of NLRP3, apoptosis-associated speck-like protein containing a CARD (ASC), and procaspase-1 into an inflammasome complex. This triggers the transformation of procaspase-1 to caspase-1, and the production and secretion of mature IL-10 and IL-18 (Kim et al., J. Inflamm. 2015, 12, 41; Ozaki et al., J. Inflamm. Res. 2015, 8, 15-27; Rabeony et al., Eur. J. Immunol. 2015, 45, 2847). During inflammasome complex assembly, the oligomerization of NLRP3 triggers the nucleation of ASC and an event commonly referred to as “ASC SPECK” formation as it is identified in the cell as a discrete puncta within the cell after staining and visualization of ASC using common immunocytochemical methods.

The ability of compounds to inhibit NLRP3 inflammasome activation was determined in vitro by monitoring formation of the ASC-SPECK in human monocytic THP-1 cells after stimulation. THP-1 cells (ATCC catalog #TIB-202) were maintained in complete growth media containing Roswell Park Memorial Institute RPMI (ATCC catalog #30-2001), 10% heat inactivated fetal bovine serum, 1× penicillin/streptomycin and 0.05 mM 2-mercaptoethanol. At the start of the assay, undifferentiated THP-1 cells were plated at a density of 20,000 cells per well in a 384-well plate (Poly-D-lysine coated Cell Carrier Ultra microplate, Perkin Elmer catalog #6057500) in complete growth media supplemented with 10 ng/ml phorbol 12-myristate 13-acetate (PMA; Sigma catalog #P8139), and then incubated overnight. The next day, media was replaced with assay media [RPMI (Gibco catalog #11875-093), 0.01% bovine serum albumin (BSA)]. Compounds were serially diluted in DMSO and then added to wells one hour prior to the addition of 12.5 ug/ml Gramicidin (Enzo Lifescience, catalog #ALX-350-233-M005). All incubations were carried out at 37° C. (5% C_{02/95}% air). Following a 3-hour treatment with gramicidin, cells are fixed with 4% paraformaldehyde and stored at 4° C. until immunofluorescence staining.

Immunofluorescence staining: Anti-ASC antibody (MBL catalog #D086-3) was desalted and labeled with Alexa 488 antibody labeling kit (Thermo catalog #A20181) prior to use as described below. After fixation, the following steps were carried out at room temperature. Cells were first permeabilized with 0.3% Triton X-100 in phosphate-buffered saline (PBS) for 15 minutes and then incubated in blocking buffer containing 5% goat serum, 0.3% tween-20 and 0.03% sodium azide in PBS for 1 hour. Cells were stained with a mixture of ASC-Alexa 488 antibody (diluted 1:200 in blocking buffer) and nuclear stain DRAQ5 (1:5000 in blocking buffer, Thermo catalog #62251) in blocking buffer for 1 hour. Following a wash with 0.3% Tween-20 in PBS, plates were imaged with an Opera Phenix High Content Screening System. The number of DRAQ5 positive cells containing ASC SPECKS were quantified in each well.

Data analysis: EC₅₀ values were calculated by standard curve-fitting analysis using an internally developed program in TIBCO Spotfire software.

The compounds of structural formula I inhibit NLRP3 inflammasome activation in the above Biological Assay and have EC₅₀ values of less than 5 micromolar. Specific EC₅₀ values of the compounds of Examples 1-22 in the above Biological Assay are listed in Table I.

TABLE I
EC₅₀ values (nM) for Examples that inhibit NLRP3 inflammasome
activation in the above Biological Assay:

| Example | EC₅₀ (nM) |
|---|---|
| 1 | 42 |
| 2 | 5 |
| 3 | 125 |
| 4 | 157 |
| 5 | 1434 |
| 6 | 118 |
| 7 | 240 |
| 8 | 285 |
| 9 | 48 |
| 10 | 380 |
| 11 | 1071 |
| 12 | 253 |
| 13 | 629 |
| 14 | 27 |
| 15 | 692 |
| 16 | 317 |
| 17 | 597 |
| 18 | 204 |
| 19 | 431 |
| 20 | 874 |
| 21 | 351 |
| 22 | 784 |

The scope of the claims should not be limited by the preferred embodiments set forth in the examples, but should be given the broadest interpretation consistent with the description as a whole.

While the disclosure has been described and illustrated with reference to certain particular embodiments thereof, those skilled in the art will appreciate that various adaptations, changes, modifications, substitutions, deletions, or additions of procedures and protocols may be made without departing from the scope of the disclosure. For example, effective dosages other than the particular dosages as set forth herein above may be applicable as a consequence of variations in responsiveness of the mammal being treated for any of the indications with the compounds of structural formula I indicated above. The specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present disclosure.

What is claimed is:

1. A compound of structural formula I: 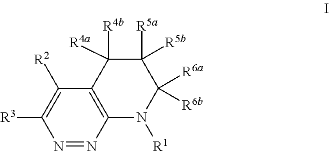
or a pharmaceutically acceptable salt thereof, wherein
R¹ is selected from the group:
1) —C₃₋₁₂cycloalkyl,
2) —C₃₋₁₂cycloalkenyl,
3) —C₂₋₁₁cycloheteroalkyl,
4) —C₂₋₁₁cycloheteroalkenyl,
5) aryl,
6) heteroaryl,
7) —C₁₋₆alkyl,
8) —C₁₋₆alkyl-OH,
9) —C₁₋₆alkyl-C₃₋₁₂cycloalkyl,
10) —C₁₋₆alkyl-C₂₋₁₁cycloheteroalkyl,
11) —C₁₋₆alkyl-C₂₋₁₁cycloheteroalkenyl,
12) —C₁₋₆alkyl-aryl, and
13) —C₁₋₆alkyl-heteroaryl,
wherein each alkyl, cycloalkyl, cycloalkenyl, cycloheteroalkyl, cycloheteroalkenyl, aryl and heteroaryl is unsubstituted or substituted with one to six substituents selected from R^{a};
R² is selected from the group:
1) hydrogen,
2) CN,
3) —CF₃,
4) —CHF2,
5) —C₁₋₆alkyl, and
6) halogen,
wherein each alkyl is unsubstituted or substituted with one to five substituents selected from R^{b};
R³ is selected from the group:
1) aryl, and
2) heteroaryl,
wherein each aryl and heteroaryl is unsubstituted or substituted with one to five substituents selected from R^{c};
R^{4a} is selected from the group:
1) hydrogen,
2) CN,
3) —C₁₋₆alkyl,
4) —O—C₁₋₆alkyl,
5) —N(R^{k})₂, and
6) halogen,
wherein each alkyl is unsubstituted or substituted with one to five substituents selected from R^{d};
R^{4b} is selected from the group:
1) hydrogen,
2) CN,
3) —C₁₋₆alkyl,
4) —O—C₁₋₆alkyl,
5) —N(R^{k})₂, and
6) halogen,
wherein each alkyl is unsubstituted or substituted with one to five substituents selected from R^{d};
R^{5a} is selected from the group:
1) hydrogen,
2) CN,
3) —C₁₋₆alkyl,
4) —O—C₁₋₆alkyl,
5) —N(R^{k})₂,
6) halogen,
7) —C₃₋₆cycloalkyl, and
8) —C₂₋₆cycloheteroalkyl,
wherein each alkyl, cycloalkyl and cycloheteroalkyl is unsubstituted or substituted with one to five substituents selected from R^{e-};
R^{5b} is selected from the group:
1) hydrogen,
2) CN,
3) —C₁₋₆alkyl,
4) —O—C₁₋₆alkyl,
5) —N(R^{k})₂,
6) halogen,
7) —C₃₋₆cycloalkyl, and
8) —C₂₋₆cycloheteroalkyl,
wherein each alkyl, cycloalkyl and cycloheteroalkyl is unsubstituted or substituted with one to five substituents selected from R^{e};
R^{6a} is selected from the group:
1) hydrogen,
2) CN,
3) —C₁₋₆alkyl, and
4) halogen,
wherein each alkyl is unsubstituted or substituted with one to five substituents selected from R^{e};
R^{6b} is selected from the group:
1) hydrogen,
2) CN,
3) —C₁₋₆alkyl, and
4) halogen,
wherein each alkyl is unsubstituted or substituted with one to five substituents selected from R^{e};
each R^{a} is independently selected from the group:
1) CN,
2) oxo,
3) —OH,
4) halogen,
5) —C₁₋₆alkyl,
6) —C₁₋₆alkyl-OH,
7) —O—C₁₋₆alkyl,
8) —C₃₋₆cycloalkyl,
9) —C₂₋₆cycloheteroalkyl,
10) aryl,
11) heteroaryl,
12) —C(O)C₁₋₆alkyl,
13) —C(O)C₃₋₆cycloalkyl,
14) —C₁₋₆alkyl-aryl,
15) —C₁₋₆alkyl-heteroaryl,
16) —C₁₋₆alkyl-C₃₋₆cycloalkyl,
17) —C₁₋₆alkyl-C₂₋₆cycloheteroalkyl,
18) —(CH₂)ₚ—O—C₁₋₆alkyl,
19) —(CH₂)ₚ—O—C₃₋₆cycloalkyl,
20) —(CH₂)ₚ—O—C₂₋₆cycloheteroalkyl,
21) —(CH₂)ₚ—O-aryl,
22) —(CH₂)ₚ—O-heteroaryl,
23) —(CH₂)ₚ—S(O)ᵣR^{f}, and
24) —N(R^{g})₂,
wherein each alkyl, cycloalkyl, cycloheteroalkyl, aryl and heteroaryl is unsubstituted or substituted with one to six substituents selected from halogen, CF₃, OH, C₁₋₆alkyl, and —OC₁₋₆-alkyl;
each R^{b} is independently selected from the group:
1) CF₃,
2) halogen,
3) —C₁₋₆alkyl, and
4) —C₃₋₆cycloalkyl;
each R^{c} is independently selected from the group:
1) CN,
2) CF₃,
3) —OH,
4) oxo,
5) halogen,
6) —C₁₋₆alkyl,
7) —O—C₁₋₆alkyl,
8) —C₃₋₆cycloalkyl,
9) —C₂₋₆cycloheteroalkyl,
10) aryl,
11) heteroaryl,
12) —C₁₋₆alkyl-aryl,
13) —C₁₋₆alkyl-heteroaryl,
14) —C₁₋₆alkyl-C₃₋₆cycloalkyl,
15) —C₁₋₆alkyl-C₂₋₆cycloheteroalkyl,
16) —(CH₂)_{q}—O—C₁₋₆alkyl,
17) —(CH₂)_{q}—O—C₃₋₆cycloalkyl,
18) —(CH₂)_{q}—O—C₂₋₆cycloheteroalkyl,
19) —(CH₂)_{q}—O-aryl,
20) —(CH₂)_{q}—O-heteroaryl,
21) —OC₁₋₆alkyl-C₃₋₆cycloalkyl,
22) —OC₁₋₆alkyl-C₂₋₆cycloheteroalkyl,
23) —OC₁₋₆alkyl-aryl,
24) —OC₁₋₆alkyl-heteroaryl,
25) —(CH₂)_{q}-S(O)ᵣR^{h},
26) —N(Rⁱ)₂,
27) —C(O)Rⁱ, and
28) —C(O)NRⁱ,
wherein each alkyl, cycloalkyl, cycloheteroalkyl, aryl, and heteroaryl is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl;
each R^{d} is independently selected from the group:
1) hydrogen,
2) OH
3) halogen, and
4) —C₁₋₆alkyl;
each R^{e} is independently selected from the group:
1) hydrogen,
2) OH,
3) halogen, and
4) —C₁₋₆alkyl;
each R^{f} is independently selected from the group:
1) hydrogen,
2) —C₁₋₆alkyl,
3) —C₃₋₆cycloalkyl, and
4) —C₂₋₆cycloheteroalkyl;
each R^{g} is independently selected from the group:
1) hydrogen,
2) —C₁₋₆alkyl,
3) —C₃₋₆cycloalkyl,
4) —C₂₋₆cycloheteroalkyl,
5) aryl,
6) heteroaryl,
7) —C(O)C₁₋₆alkyl, and
8) —S(O)ᵣR^{f},
wherein alkyl, cycloalkyl, cycloheteroalkyl, aryl and heteroaryl is unsubstituted or substituted with one to three substituents selected from: CF₃, halogen, OH and —OC₁₋₆alkyl;
each R^{h} is independently selected from the group:
1) hydrogen,
2) —C₁₋₆alkyl,
3) —C₃₋₆cycloalkyl, and
4) —C₂₋₆cycloheteroalkyl;
each Rⁱ is independently selected from the group:
1) hydrogen,
2) —C₁₋₆alkyl,
3) —C₃₋₆cycloalkyl, and
4) —C₂₋₆cycloheteroalkyl;
each R^{j} is independently selected from the group:
1) OH,
2) —C₁₋₆alkyl,
3) —C₃₋₆cycloalkyl, and
4) —C₂₋₆cycloheteroalkyl,
wherein alkyl, cycloalkyl and cycloheteroalkyl is unsubstituted or substituted with one to three substituents selected from: CF₃, halogen, OH and —OC₁₋₆alkyl;
each R^{k} is independently selected from the group:
1) hydrogen, and
2) —C₁₋₆alkyl;
p is 0, 1, 2, 3, 4, 5 or 6;
q is 0, 1, 2, 3, 4, 5 or 6; and
r is 1 or 2.

2. The compound according to claim 1 wherein R¹ is selected from the group:
1) —C₃₋₁₂cycloalkyl,
2) —C₂₋₁₁cycloheteroalkyl, and
3) —C₁₋₆alkyl-C₂₋₁₁cycloheteroalkyl,
wherein each alkyl, cycloalkyl and cycloheteroalkyl is unsubstituted or substituted with one to six substituents selected from R^{a};
or a pharmaceutically acceptable salt thereof.

3. The compound according to claim 1 wherein
R¹ is selected from the group:
1) cyclobutane,
2) cyclohexane,
3) bicyclo[3.1.1]heptane,
4) piperidine, and
5) —CH₂-pyrrolidine,
wherein cyclobutane, cyclohexane, bicyclo[3.1.1]heptane, piperidine and —CH₂-pyrrolidine is unsubstituted or substituted with one to six substituents selected from R^{a};
or a pharmaceutically acceptable salt thereof.

4. The compound according to claim 1 wherein
R¹ is selected from the group:
1) —C₃₋₁₂cycloalkyl, and
2) —C₂₋₁₁cycloheteroalkyl,
wherein each cycloalkyl and cycloheteroalkyl is unsubstituted or substituted with one to six substituents selected from R^{a};
or a pharmaceutically acceptable salt thereof.

5. The compound according to claim 1 wherein
R¹ is selected from the group:
1) cyclobutane,
2) cyclohexane,
3) bicyclo[3.1.1]heptane, and
4) piperidine,
wherein cyclobutane, cyclohexane, bicyclo[3.1.1]heptane, and piperidine is unsubstituted or substituted with one to six substituents selected from R^{a};
or a pharmaceutically acceptable salt thereof.

6. The compound according to claim 1 wherein
R² is selected from the group:
1) hydrogen, and
2) —C₁₋₆alkyl,
wherein alkyl is unsubstituted or substituted with one to five substituents selected from R^{b};
or a pharmaceutically acceptable salt thereof.

7. The compound according to claim 1 wherein
R³ is selected from the group:
1) phenyl,
2) benzofuran, and
3) pyridine,
wherein phenyl, benzofuran and pyridine is unsubstituted or substituted with one to five substituents selected from R^{c};
or a pharmaceutically acceptable salt thereof.

8. The compound according to claim 1 wherein
R³ is aryl, wherein aryl is unsubstituted or substituted with one to five substituents selected from R^{c};
or a pharmaceutically acceptable salt thereof.

9. The compound according to claim 1 wherein
R³ is phenyl, wherein phenyl is unsubstituted or substituted with one to five substituents selected from R^{c};
or a pharmaceutically acceptable salt thereof.

10. The compound according to claim 1 wherein
R^{4a} is selected from the group:
1) hydrogen, and
2) —C₁₋₆alkyl,
wherein each alkyl is unsubstituted or substituted with one to five substituents selected from R^{d};
R^{4b} is selected from the group:
1) hydrogen, and
2) —C₁₋₆alkyl,
wherein each alkyl is unsubstituted or substituted with one to five substituents selected from R^{d};
R^{5a} is selected from the group:
1) hydrogen, and
2) —N(R^{k})₂;
R^{5b} is selected from the group:
1) hydrogen, and
2) —N(R^{k})₂;
R^{6a} is selected from the group:
1) hydrogen, and
2) —C₁₋₆alkyl,
wherein each alkyl is unsubstituted or substituted with one to five substituents selected from R^{e}; and
R^{6b} is selected from the group:
1) hydrogen, and
2) —C₁₋₆alkyl,
wherein each alkyl is unsubstituted or substituted with one to five substituents selected from R^{e};
or a pharmaceutically acceptable salt thereof.

11. The compound according to claim 1 wherein
R^{4a} is hydrogen;
R^{4b} is hydrogen;
R^{5a} is hydrogen.
R^{5b} is hydrogen;
R^{6a} is hydrogen; and
R^{6b} is hydrogen;
or a pharmaceutically acceptable salt thereof.

12. The compound according to claim 1 wherein
R¹ is selected from the group:
1) —C₃₋₁₂cycloalkyl,
2) —C₂₋₁₁cycloheteroalkyl, and
3) —C₁₋₆alkyl-C₂₋₁₁cycloheteroalkyl,
wherein each alkyl, cycloalkyl and cycloheteroalkyl is unsubstituted or substituted with one to six substituents selected from R^{a};
R² is selected from the group:
1) hydrogen, and
2) —C₁₋₆alkyl,
wherein alkyl is unsubstituted or substituted with one to five substituents selected from R^{b};
R³ is selected from the group:
1) aryl, and
2) heteroaryl,
wherein each aryl and heteroaryl is unsubstituted or substituted with one to five substituents selected from R^{c};
R^{4a} is selected from the group:
1) hydrogen, and
2) —C₁₋₆alkyl,
wherein each alkyl is unsubstituted or substituted with one to five substituents selected from R^{d};
R^{4b} is selected from the group:
1) hydrogen, and
2) —C₁₋₆alkyl,
wherein each alkyl is unsubstituted or substituted with one to five substituents selected from R^{d};
R^{5a} is selected from the group:
1) hydrogen,
2) CN,
3) —C₁₋₆alkyl,
4) —O—C₁₋₆alkyl,
5) —N(R^{k})₂, and
6) halogen,
wherein each alkyl is unsubstituted or substituted with one to five substituents selected from R^{e};
R^{5b} is selected from the group:
1) hydrogen,
2) CN,
3) —C₁₋₆alkyl,
4) —O—C₁₋₆alkyl,
5) —N(R^{k})₂, and
6) halogen,
wherein each alkyl is unsubstituted or substituted with one to five substituents selected from R^{e};
R^{6a} is selected from the group:
1) hydrogen, and
2) —C₁₋₆alkyl,
wherein each alkyl is unsubstituted or substituted with one to five substituents selected from R^{e};
R^{6b} is selected from the group:
1) hydrogen, and
2) —C₁₋₆alkyl,
wherein each alkyl is unsubstituted or substituted with one to five substituents selected from R^{e};
or a pharmaceutically acceptable salt thereof.

13. The compound according to claim 1 wherein
R¹ is selected from the group:
1) cyclobutane,
2) cyclohexane,
3) bicyclo[3.1.1]heptane,
4) piperidine, and
5) —CH₂-pyrrolidine,
wherein cyclobutane, cyclohexane, bicyclo[3.1.1]heptane, piperidine, and —CH₂-pyrrolidine is unsubstituted or substituted with one to six substituents selected from R^{a};
R² is selected from the group:
1) hydrogen, and
2) —C₁₋₆alkyl,
wherein alkyl is unsubstituted or substituted with one to five substituents selected from R^{b};
R³ is selected from the group:
1) phenyl,
2) benzofuran, and
3) pyridine,
wherein phenyl, benzofuran and pyridine is unsubstituted or substituted with one to five substituents selected from R^{c};
R^{4a} is selected from the group:
1) hydrogen, and
2) —C₁₋₆alkyl,
wherein each alkyl is unsubstituted or substituted with one to five substituents selected from R^{d};
R^{4b} is selected from the group:
1) hydrogen, and
2) —C₁₋₆alkyl,
wherein each alkyl is unsubstituted or substituted with one to five substituents selected from R^{d};
R^{5a} is selected from the group:
1) hydrogen,
2) CN,
3) —C₁₋₆alkyl,
4) —O—C₁₋₆alkyl,
5) —N(R^{k})₂, and
6) halogen,
wherein each alkyl is unsubstituted or substituted with one to five substituents selected from R^{e};
R^{5b} is selected from the group:
1) hydrogen,
2) CN,
3) —C₁₋₆alkyl,
4) —O—C₁₋₆alkyl,
5) —N(R^{k})₂, and
6) halogen,
wherein each alkyl is unsubstituted or substituted with one to five substituents selected from R^{e};
R^{6a} is selected from the group:
1) hydrogen, and
2) —C₁₋₆alkyl,
wherein each alkyl is unsubstituted or substituted with one to five substituents selected from R^{e};
R^{6b} is selected from the group:
1) hydrogen, and
2) —C₁₋₆alkyl,
wherein each alkyl is unsubstituted or substituted with one to five substituents selected from R^{e};
or a pharmaceutically acceptable salt thereof.

14. The compound according to claim 1 wherein
R¹ is selected from the group:
1) —C₃₋₁₂cycloalkyl, and
2) —C₂₋₁₁cycloheteroalkyl,
wherein each cycloalkyl and cycloheteroalkyl is unsubstituted or substituted with one to six substituents selected from R^{a};
R² is selected from the group:
1) hydrogen, and
2) —C₁₋₆alkyl,
wherein alkyl is unsubstituted or substituted with one to five substituents selected from R^{b};
R³ is aryl, wherein aryl is unsubstituted or substituted with one to five substituents selected from R^{c};
R^{4a} is selected from the group:
1) hydrogen, and
2) —C₁₋₆alkyl,
wherein each alkyl is unsubstituted or substituted with one to five substituents selected from R^{d};
R^{4b} is selected from the group:
1) hydrogen, and
2) —C₁₋₆alkyl,
wherein each alkyl is unsubstituted or substituted with one to five substituents selected from R^{d};
R^{5a} is selected from the group:
1) hydrogen, and
2) —N(R^{k})₂;
R^{5b} is selected from the group:
1) hydrogen, and
2) —N(R^{k})₂;
R^{6a} is selected from the group:
1) hydrogen, and
2) —C₁₋₆alkyl,
wherein each alkyl is unsubstituted or substituted with one to five substituents selected from R^{e};
R^{6b} is selected from the group:
1) hydrogen, and
2) —C₁₋₆alkyl,
wherein each alkyl is unsubstituted or substituted with one to five substituents selected from R^{e};
or a pharmaceutically acceptable salt thereof.

15. The compound according to claim 1 wherein
R¹ is selected from the group:
1) cyclobutane,
2) cyclohexane,
3) bicyclo[3.1.1]heptane, and
4) piperidine,
wherein cyclobutane, cyclohexane, bicyclo[3.1.1]heptane, and piperidine is unsubstituted or substituted with one to six substituents selected from R^{a};
R² is selected from the group:
1) hydrogen, and
2) —C₁₋₆alkyl,
wherein alkyl is unsubstituted or substituted with one to five substituents selected from R^{b};
R³ is phenyl, wherein phenyl is unsubstituted or substituted with one to five substituents selected from R^{c};
R^{4a} is hydrogen;
R^{4b} is hydrogen;
R^{5a} is hydrogen.
R^{5b} is hydrogen;
R^{6a} is hydrogen; and
R^{6b} is hydrogen;
or a pharmaceutically acceptable salt thereof.

16. The compound according to claim 1 selected from:
1) 2-(8-((1s,3s)-3-hydroxy-3-methylcyclobutyl)-5,6,7,8-tetrahydropyrido[2,3-c]pyridazin-3-yl)-3-methyl-5-(trifluoromethyl)phenol;
2) (R)-2-(8-(1-ethylpiperidin-3-yl)-5,6,7,8-tetrahydropyrido[2,3-c]pyridazin-3-yl)-3-methyl-5-(trifluoromethyl)phenol;
3) 5-(3-(2-hydroxy-6-methyl-4-(trifluoromethyl)phenyl)-6,7-dihydropyrido[2,3-c]pyridazin-8(5H)-yl)bicyclo[3.1.1]heptan-1-ol;
4) 2-(8-((1R,2R)-2-hydroxycyclohexyl)-5,6,7,8-tetrahydropyrido[2,3-c]pyridazin-3-yl)-3-methyl-5-(trifluoromethyl)phenol;
5) 2-(8-((1S,2R or 1R,2S)-2-hydroxy-3,3-dimethylcyclobutyl)-5,6,7,8-tetrahydropyrido[2,3-c]pyridazin-3-yl)-3-methyl-5-(trifluoromethyl)phenol;
6) 2-(8-((1R,2S or 1S,2R)-2-hydroxy-3,3-dimethylcyclobutyl)-5,6,7,8-tetrahydropyrido[2,3-c]pyridazin-3-yl)-3-methyl-5-(trifluoromethyl)phenol;
7) 2-(8-(((2S,4S)-4-fluoropyrrolidin-2-yl)methyl)-5,6,7,8-tetrahydropyrido[2,3-c]pyridazin-3-yl)-3-methyl-5-(trifluoromethyl)phenol;
8) 2-((R)-6-amino-8-((1s,3S)-3-hydroxy-3-methylcyclobutyl)-5,6,7,8-tetrahydropyrido[2,3-c]pyridazin-3-yl)-3-methyl-5-(trifluoromethyl)phenol;
9) (R)-2-(8-(1-ethylpiperidin-3-yl)-4-methyl-5,6,7,8-tetrahydropyrido[2,3-c]pyridazin-3-yl)-5-(trifluoromethyl)phenol;
10) (R)-3-(2-(difluoromethoxy)-4-(trifluoromethyl)phenyl)-8-(1-ethylpiperidin-3-yl)-4-methyl-5,6,7,8-tetrahydropyrido[2,3-c]pyridazine;
11) (R)-3-(2-(difluoromethoxy)-6-(trifluoromethyl)pyridin-3-yl)-8-(1-ethylpiperidin-3-yl)-4-methyl-5,6,7,8-tetrahydropyrido[2,3-c]pyridazine;
12) (R)-3-(benzofuran-5-yl)-8-(1-ethylpiperidin-3-yl)-4-methyl-5,6,7,8-tetrahydropyrido[2,3-c]pyridazine;
13) 2-(8-(((2S,4S)-4-fluoro-1-methylpyrrolidin-2-yl)methyl)-5,6,7,8-tetrahydropyrido[2,3-c]pyridazin-3-yl)-3-methyl-5-(trifluoromethyl)phenol;
14) (3S,4R)-3-(3-(2-hydroxy-6-methyl-4-(trifluoromethyl)phenyl)-6,7-dihydropyrido[2,3-c]pyridazin-8(5H)-yl)-1-methylpiperidin-4-ol;
15) 2-(8-(((2S,4S)-1-ethyl-4-fluoropyrrolidin-2-yl)methyl)-5,6,7,8-tetrahydropyrido[2,3-c]pyridazin-3-yl)-3-methyl-5-(trifluoromethyl)phenol;
16) 1-((2S,4R)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)ethan-1-one;
17) 1-((2S,4S)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)ethan-1-one;
18) 1-((2S,4R)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)propan-1-one;
19) cyclopropyl((2S,4R)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)methanone;
20) cyclobutyl((2S,4R)-4-fluoro-2-((3-(2-hydroxy-6-methyl-4-(trifluoromethyl)phenyl)-6,7-dihydropyrido[2,3-c]pyridazin-8(5H)-yl)methyl)pyrrolidin-1-yl)methanone;
21) 2-(8-(((2S,4R)-4-fluoro-1-(methylsulfonyl)pyrrolidin-2-yl)methyl)-5,6,7,8-tetrahydropyrido[2,3-c]pyridazin-3-yl)-3-methyl-5-(trifluoromethyl)phenol; and
22) 2-(8-(((2S,4S)-4-fluoro-1-(methylsulfonyl)pyrrolidin-2-yl)methyl)-5,6,7,8-tetrahydropyrido[2,3-c]pyridazin-3-yl)-3-methyl-5-(trifluoromethyl)phenol;
or a pharmaceutically acceptable salt thereof.

17. A pharmaceutical composition comprising a compound of claim 1, or a pharmaceutically acceptable salt thereof, and a pharmaceutically acceptable carrier.

18. (canceled)

19. (canceled)

20. (canceled)

21. (canceled)

22. (canceled)

23. A compound according to claim 1, or a pharmaceutically acceptable salt thereof, for use in therapy.

24. A method of treating or preventing a disorder, condition or disease that is responsive to the inhibition of NLRP3 in a patient in need thereof comprising administration of a therapeutically effective amount of a compound according to claim 1, or a pharmaceutically acceptable salt thereof.

25. The method of claim 24 wherein the disorder is selected from: an inflammatory disorder, a fibrotic disorder, a cardiovascular disorder, a metabolic disorder and a neurodegenerative disorder.

26. The method of claim 25 wherein the disorder is an inflammatory disorder.

27. The method of claim 26 wherein the inflammatory disorder is selected from: an auto-immune disorder, an auto-inflammatory disorder, an inflammatory joint disorder, an inflammatory skin disorder, and a neuroinflammatory disorder.

28. The method of claim 24 wherein the disorder is selected from: atherosclerosis, non-alcoholic steatohepatitis, Alzheimer's disease and Parkinson's disease.
